(19) United States
(12) Patent Application Publication
(10) Pub. No.: US 2023/0277597 A1
(43) Pub. Date: Sep. 7, 2023

(54) HAPLOIDENTICAL MIXED CHIMERISM FOR TREATING AUTOIMMUNE DISEASES

(71) Applicant: CITY OF HOPE, Duarte, CA (US)

(72) Inventor: Defu ZENG, Arcadia, CA (US)

(73) Assignee: CITY OF HOPE, Duarte, CA (US)

(21) Appl. No.: 18/170,913

(22) Filed: Feb. 17, 2023

Related U.S. Application Data
(63) Continuation of application No. PCT/US2021/046339, filed on Aug. 17, 2021.
(60) Provisional application No. 63/067,251, filed on Aug. 18, 2020.

(51) Int. Cl. A61K 35/28; A61K 31/675; A61K 31/7056; A61K 39/395; A61P 37/06

(52) U.S. Cl. CPC A61K 35/28; A61K 31/675; A61K 31/7056; A61K 39/3955; A61P 37/06

(57) ABSTRACT

Disclosed are methods of treating or preventing autoimmune diseases by inducing haploidentical mixed chimerism and condition regimen for by inducing haploidentical mixed chimerism.

### PRIORITY CLAIM

This application is a continuation of United States Patent Application No. PCT/US2021/046339, filed Aug. 17, 2021, which claims the benefit of U.S. Provisional Patent Application No. 63/067,251, filed Aug. 18, 2020, the contents of which are hereby incorporated by reference in their entirety, including drawings.

### BACKGROUND

Haploidentical hematopoietic cell transplantation (Haplo-HCT) has been widely applied to treating hematological malignancies and non-malignant disorders (1). Induction of haploidentical mixed chimerism for organ transplantation immune tolerance is under clinical trials (NCT03292445, NCT01165762, NCT01780454, NCT02314403, NCT00801632, NCT01758042), and the results are promising (2-5). However, it remains unclear whether induction of haploidentical mixed chimerism can reverse autoimmunity, because induction of MHC-matched or HLA-matched mixed chimerism is not able to reverse autoimmunity in T1 D mice or systemic lupus in humans (6-8). Therefore, there is a need to further explore the effects of haploidentical mixed chimerism in patients, particularly, in patients receiving transplantation and/or patients suffering from autoimmunity.

### SUMMARY

In one aspect, disclosed herein is a conditioning regimen for inducing haploidentical mixed chimerism in a subject comprising administration of radiation-free, non-myeloablative low doses of cyclophosphamide (CY), pentostatin (PT), and anti-thymocyte globulin (ATG), and administration of a population of CD4⁺ T-depleted hematopoietic cells from a donor. In some embodiments, the donor CD4⁺ T-depleted hematopoietic cells include donor CD4⁺ T-depleted spleen cells, and donor CD4⁺ T-depleted bone marrow cells. In some embodiments, the donor CD4⁺ T-depleted hematopoietic cells are CD4⁺ T-depleted G-CSF-mobilized blood mononuclear cells comprising donor hematopoietic stem cells and CD8⁺ T cells. In some embodiments, the donor is haploidentical to the subject. In some embodiments, the donor is haplo-mismatched to the subject. In some embodiments, the donor is not full-HLA- or MHC-matched to the subject. In some embodiments, the donor CD4⁺ T-depleted hematopoietic cells can be administered on the same day as, before, or after the administration of CY, PT and ATG. In some embodiments, the subject is a mammal such as human.

In another aspect, this disclosure relates to a method of inducing haploidentical mixed chimerism in a subject by administering to the subject radiation-free, non-myeloablative low doses of CY, PT and ATG, and administering to the subject a population of CD4⁺ T-depleted hematopoietic cells from a donor. In some embodiments, the donor CD4⁺ T-depleted hematopoietic cells include donor CD4⁺ T-depleted spleen cells, and donor CD4⁺ T-depleted bone marrow cells. In some embodiments, the donor CD4⁺ T-depleted hematopoietic cells are CD4⁺ T-depleted G-CSF-mobilized blood mononuclear cells comprising donor hematopoietic stem cells and CD8⁺ T cells. In some embodiments, the donor is haploidentical to the subject. In some embodiments, the donor is haplo-mismatched to the subject. In some embodiments, the donor is not full-HLA- or MHC-matched to the subject. The donor CD4⁺ T-depleted hematopoietic cells can be administered on the same day as, before, or after the administration of CY, PT and ATG. In some embodiments, the subject is a mammal such as human.

In yet another aspect, this disclosure relates to a method of treating or preventing the onset of an autoimmune disease in a subject by inducing haploidentical mixed chimerism in the subject. The method entails administering to the subject radiation-free, non-myeloablative low doses of CY, PT and ATG, and administering to the subject a population of CD4⁺ T-depleted hematopoietic cells from a donor. In some embodiments, the donor CD4⁺ T-depleted hematopoietic cells include donor CD4⁺ T-depleted spleen cells, and donor CD4⁺ T-depleted bone marrow cells. In some embodiments, the donor CD4⁺ T-depleted hematopoietic cells are CD4⁺ T-depleted G-CSF-mobilized blood mononuclear cells comprising donor hematopoietic stem cells and CD8⁺ T cells. In some embodiments, the donor is haploidentical to the subject. In some embodiments, the donor is haplo-mismatched to the subject. In some embodiments, the donor is not full-HLA- or MHC-matched to the subject. The donor CD4⁺ T-depleted hematopoietic cells can be administered on the same day as, before, or after the administration of CY, PT and ATG. In some embodiments, the subject is a mammal such as human. In some embodiments, the subject suffers from or at an elevated risk of suffering from an autoimmune disease, including but not limited to, multiple sclerosis, type-1 diabetes, systemic lupus, scleroderma, chronic graft versus host disease, aplastic anemia, and arthritis.

### BRIEF DESCRIPTION OF THE DRAWINGS

FIG. 1 illustrates the mechanism of induction of MHC-haploidentical mixed chimerism (Haplo-MC). Induction of Haplo-MC augments thymic negative selection of Tcon and production of donor- and host-type tTreg cells, leading to re-establishment of central tolerance. In the periphery, donor- and host-type tTreg cells interact with host-type DCs such as pDCs and restore their tolerogenic features such as upregulation of PD-L1 expression. PD-L1 on DCs interact with PD-1 on activated host-type autoreactive T cells and augment the T cell differentiation into antigen-specific Treg cells. All tTreg and pTreg cells and tolerogenic DCs work together to maintain tolerance status of residual host-type autoreactive T cells.

FIGS. 2A-2C show that Haplo-MC status was achieved in WT NOD mice with haploidentical donors. Prediabetic 9-12 weeks old NOD mice were conditioned with ATG+CY+PT, and transplanted with BM (50×10⁶) and SPL cells (30×10⁶) from H-2b/g7 F1 or H-2s/g7 F1 donors respectively, and co-injected with depleting anti-CD4 mAb (500 μg/mouse). The recipients were monitored for chimerism in the peripheral blood and levels of blood glycose. FIG. 2A shows a representative flow cytometry pattern of T cells (TCRβ⁺), B cells (B220+), and myeloid cells (Mac1/Gr1+) in the peripheral blood at 6 weeks after HCT and mean±SE of percentage of donor- and host-type cells of 5-7 representative mice for 12 mice in each group combined from two replicate experiments. FIGS. 2B and 2C show that spleen (2B) and bone marrow (2C) samples from chimeric WT NOD or conditioning alone control were collected at day 100 for validating the chimerism status. One representative flow cytometry pattern and mean±SE of percentage of 5 representative mice in each group are shown for total 12 mice from two replicate experiments.

FIGS. 3A and 3B show that no sign of clinical or tissue GVHD was observed in Haplo-MC WT NOD mice. Bodyweight of WT NOD mice in FIG. 4 was monitored for 100 days after HCT. At D100, Liver and lung samples were collected and subjected to HE staining to evaluate GVHD histopathology. FIG. 3A: Body weight curve of 12 mice is shown. FIG. 3B: One representative liver and lung tissue microphoto is shown of 5 mice examined in each group.

FIGS. 4A-4F show that induction of Haplo-MC prevented diabetes onset and reversed new-onset T1 D in WT NOD mice, with clearing up insulitis. Prediabetic 9-12 weeks old NOD and new-onset diabetic NOD mice were conditioned with ATG+CY+PT, and transplanted with BM (50×10⁶) and SPL cells (30×10⁶) from H-2^{g/7} F1 or H-2^{s/g7} F1 donors, respectively, and co-injected with depleting anti-CD4 mAb (500 μg/mouse). Recipients were monitored for diabetes development for 100 days after HCT. FIG. 4A: T1 D development curves in prediabetic NOD mice (n=20-37 from >3 experiments). P<0.0001 when comparing conditioning alone control to either H-2b/g7 or H-2s/g7 chimera using log-rank test. FIGS. 4B and 4C: 100 days after HCT, residual non-diabetic mice were subject to insulitis evaluation. Representative HE histopathology photomicrographs are shown. Summary insulitis score is shown in mean (n=9-12). FIG. 4D: T1 D relapse curves of new-onset diabetic NOD mice given conditioning alone or induction of either H-2^{g/7} or H-2^{s/g7} Haplo-MC (n=12-24 from >3 experiments). FIGS. 4E and 4F: Representative photomicrographs and summary (mean) of insulitis score of recipients with normal glycemia 100 days after HCT or control mice given conditioning alone (n=6-12). Statistic comparison of insulitis was completed by using Chi-square test (4B and 4F) (****P<0.0001).

FIGS. 5A-5C show that Haplo-MC was achieved in thymectomized WT NOD mice. WT-NOD mice were given thymectomy at age of 6-week by JAX lab. 3-4 weeks after thymectomy, mice were conditioned with ATG+CY+PT and transplanted with BM (50×10⁶) from H-2^{s/g7} F1 donors. Recipients were monitored for chimerism in the blood and levels of blood glucose for up to 80 days after HCT. At the end of experiments, recipients were validated for mixed chimerism status of T cells (TCRβ⁺), B cells (B220⁺), and myeloid cells (Mac1/Gr1⁺) in the peripheral blood (5A), spleen (5B) and BM (5C). One representative flow cytometry pattern and mean±SE of percentage of 5-7 representative mice are shown for totally 10 mice in two replicate experiments.

FIGS. 6A-6C show that Haplo-MC prevented T1 D development and eliminate insulitis in thymectomized WT NOD mice. The same thymectomized NOD mice with Haplo-MC described in FIG. 5 were monitored for T1 D development and evaluated for insulitis at the end of experiments. FIG. 6A: T1 D development curves, 10 mice/group combined from two replicate experiments. FIGS. 6B-6C: Insulitis score and representative insulitis microphotos of mice that did not show hyperglycemia by the end of experiments are shown for 4-6 mice examined in each group.

FIGS. 7A-7C show that Haplo-MC status was achieved in lethal TBI-conditioned WT NOD mice. Prediabetic 9-12 weeks old NOD mice were conditioned with lethal TBI (950 cGy) and transplanted with syngeneic TCD-BM (5×10⁶) from NOD mice and haplo-TCD-BM (7.5×10⁶) from H-2^{g/7} or H-2^{s/g7} F1 donors. Control recipients were transplanted with TCD-BM from NOD mice only. Recipients were monitored for chimerism in the peripheral blood and levels of blood glycose for 80 days after HCT. At the end of experiments, recipients were validated for chimerism status of T cells (TCRβ⁺), B cells (B220⁺), and myeloid cells (Mac1/Gr1⁺) in the peripheral blood (7A), spleen (7B) and BM (7C). One representative flow cytometry pattern and mean±SE of percentage of 7 representative mice are shown for total 10-15 mice from two replicate experiments.

FIGS. 8A-8C show that induction of Haplo-MC in lethal TBI-conditioned mice did not eliminate insulitis although prevented clinical T1 D. Lethal TBI-conditioned WT NOD mice were induced to developed Haplo-MC and monitored for T1 D development as described in FIG. 7. Recipients were monitored for diabetes development for 80 days after HCT. FIG. 8A: T1 D development curves in prediabetic NOD mice. There were 10-15 mice combined from two replicate experiments. FIGS. 8B-8C: 80 days after HCT, residual non-diabetic mice were subjected to insulitis evaluation. Summary insulitis score and representative islet microphotographs (magnification 10×) are shown for 5-10 representative mice from two replicate experiments.

FIGS. 9A-9C show that Haplo-MC reduced host-type CD4⁺CD8⁺ thymocytes and thymocytes with dual TCRs. 60 days after HCT, thymocytes from mixed chimeric WT NOD and BDC2.5 NOD or control mice given conditioning alone were analyzed for donor- and host-type CD4⁺CD8⁺ thymocytes. FIGS. 9A and 9B: thymocytes of WT NOD and BDC2.5 NOD are shown for donor- and host-type CD4⁺CD8⁺, respectively. n=6-15. FIG. 9C: The BDC2.5 transgenic TCR consisted of Vα1 and Vβ4. If a Vβ4⁺ T cell also expresses any Vα chain other than Vα1, such as Vα2, it is considered as a T cell expressing more than one set of TCR. Representative staining and summary (mean±SEM) of % T cells with dual TCRs among host type CD4⁺CD8⁻ population in BDC2.5 thymus are shown, n=5-7. P values were calculated using unpaired 2-tailed Student's t tests (9A and 9B) or one-way ANOVA (9C) (*p<0.05, **p<0.01, ***p<0.001, ****p<0.0001).

FIG. 10 shows that Haplo-MC status was achieved in BDC2.5 NOD mice with haploidentical donors. 6-9 weeks old BDC2.5 NOD mice were conditioned with ATG+CY+PT, and transplanted with BM (50×10⁶) and SPL cells (30×10⁶) from H-2^{b/g7} F1 or H-2^{s/g7} F1 donors, respectively, co-injected with depleting anti-CD4 mAb (500 μg/mouse). The recipients were monitored for mixed chimerism in the peripheral blood and glycose levels of blood. Haplo-MC status of T, B, and myeloid cells was validated with spleen and bone marrow MNC at the end of experiments at 60 days after HCT. One representative flow cytometry pattern and mean±SE of percentage of 5-7 representative mice in each group are shown for 10 mice from two replicate experiments. The T1 D development curve is shown in FIG. 32.

FIGS. 11A-11C show that Haplo-MC increased Treg production in thymus, with engraftment of donor type DC subsets. 60 days after HCT, H-2^{b/g7} and H-2^{s/g7} Haplo-MC and control mice were measured for host-type Foxp3⁺ Treg cells among CD4⁺CD8⁻ (CD4 SP) or CD4⁺CD8⁺ (DP) thymocytes as well as measured for donor-type DC subsets. FIG. 11A: % Treg among host-type CD4⁺ SP and DP thymocytes in WT NOD (n=7-9). FIG. 11B: % Treg among host-type CD4⁺ SP thymocytes in BDC2.5 NOD (n=7-9). FIG. 11C: % Donor-type thymic DC subset among donor-type CD11c⁺ DCs, in comparison to healthy donor controls of each strain, n=6 per group. Representative patterns and summary of mean±SEM are shown. P values were calculated using unpaired 2-tailed Student's t tests (11C) or one-way ANOVA (11A and 11B) (*p<0.05, **p<0.01, ***p<0.001).

FIGS. 12A-12B show an increase of donor-type tTreg production in thymus in transgenic BDC2.5 but not in WT NOD Haplo-MC. 60 days after HCT, H-2^{b/g7} and H-2^{s/g7} Haplo-MC mice of WT NOD (FIG. 12A) and BDC2.5 NOD (FIG. 12B) and control donor mice were measured for donor-type Foxp3⁺ Treg cells among CD4⁺CD8⁻ (CD4 SP) cells or CD4⁺CD8⁺ (DP) cells in the thymus. Representative flow cytometry patterns and mean±SEM of tTreg percentage among donor-type SP or DP thymocytes are shown for 5-7 mice for in each group from two replicate experiments. *p<0.05.

FIGS. 13A-13D show that Haplo-MC in NOD mice reduced host-type autoreactive effector memory T cells in the pancreas of WT and BDC2.5 NOD mice. 60-80 days after HCT, mononuclear cells (MNC) of spleen, pancreatic LN and pancreas of mixed chimeric or control WT and BDC2.5 NOD mice were analyzed by flow cytometry for host-type CD44^{hi}CD62L⁻CD4⁺ or CD8⁺ Tem cells. Mean±SEM of percentage and yield of CD62L⁻CD44^{hi} Tem in the Spleen (SPL), pancreatic LN (PancLN), and pancreas are shown. FIGS. 13A and 13B: CD4⁺ and CD8⁺ Tcons of WT NOD with Haplo-MC or given conditioning alone, n=5-12. FIG. 13C: Percentage and yield of CD62L⁻CD44^{hi}CD4⁺ Tem cells in BDC2.5 NOD mice, n=4-7. FIG. 13D: Percentage of antigen-specific autoreactive T cells in the pancreas of WT NOD mice. The pancreatic MNC of Haplo-MC or control WT NOD mice were stained with I-A^{g7}-HIP 2.5 tetramer to identify antigen-specific autoreactive CD4⁺ T cells or H-2^{d}-NRP-V7 tetramer to identify autoreactive CD8⁺ T cells. Representative flow cytometry patterns and mean±SEM of percentage of tetramer⁺CD4⁺ or CD8⁺ T cells are shown, n=5-11. P values were calculated using one-way ANOVA (*p<0.05, **p<0.01, ***p<0.001).

FIGS. 14A-14C show that Haplo-MC reduced host-type autoreactive CD4⁺ and CD8⁺ T effector cells in WT and BDC2.5 NOD mice. 60 days after HCT, MNC of SPL, PancLN and pancreas from WT and BDC2.5 mixed chimeras and control mice were analyzed with flow cytometry for percentage of CD45.1⁺ host-type T effector cells (CD45.1⁺CD44^{hi}CD62L⁻ TCRβ⁺). One representative pattern is shown for host type CD4⁺ Tcon in WT NOD mixed chimeras (14A), CD8⁺ T cells in WT NOD mixed chimeras(14B), and host-type CD4⁺ Tcon in BDC 2.5 NOD mixed chimeras (14C).

FIGS. 15A-15B show that Haplo-MC reduced host-type T effector memory cells in thymectomized WT NOD mice. Thymectomized NOD mice with or without induction of Haplo-MC described in FIG. 5 were further analyzed for residual host-type T cell subset at the end of experiments. Mononuclear cells (MNC) of spleen and pancreatic LN of mice with Haplo-MC, mice given conditioning only and mice given no treatment were analyzed by flow cytometry for percentage of host-type CD44^{hi}CD62L⁻ CD4⁺ T (15A) or CD8⁺ T (15B) T effector memory cells. A representative flow cytometry pattern and mean±SE of percentage and yield of CD44^{hi}CD62L⁻ effector memory T cells are shown of 5-10 representative mice in each group from two replicate experiments. *p<0.05, **p<0.01, ***p<0.001, ****p<0.0001.

FIGS. 16A-16B show that Haplo-MC increased percentage of total CD73^{hi}FR4^{hi}anergic CD4⁺ T cells and Nrp-1⁺CD73^{hi}FR4^{hi} anergic cells among host-type CD44^{hi}CD62L⁻CD4⁺ Tem cells. 60-80 days after HCT, samples of pancreatic LN and pancreas MNC were analyzed by flow cytometry for their expression of CD45.2 (donor-marker), TCRβ, CD4, Foxp3, CD62L, CD44, CD73, FR4 and Nrp-1. Representative patterns of flow cytometry and mean±SEM of percentage of CD73^{hi}FR4^{hi} anergic cells among total host-type Foxp3⁻CD62L⁻CD44^{hi}CD4⁺ Tem cells and percentage of Nrp-1⁺CD73^{hi}FR4^{hi} cells among total CD73^{hi}FR4^{hi} anergic cells are shown, n=4-8. P values were calculated using one-way ANOVA (*p<0.05, **p<0.01, ***p<0.001, **** p<0.0001).

FIG. 17 shows that Haplo-MC in thymectomized NOD mice did not increase Nrp-1⁺ cells among host-type residual CD73⁺FR4⁺ anergic CD4⁺ Tem cells. Thymectomized NOD mice with or without induction of Haplo-MC described in FIG. 5 were further analyzed for anergy status of residual host-type T cells. MNC from PancLN of mice with Haplo-MC, mice given conditioning only, and mice given no treatment were analyzed by flow cytometry for their expression of CD45.1(host-marker), TCRβ, CD4, Foxp3, CD62L, CD44, CD73, FR4 and Nrp-1. Representative patterns of flow cytometry and mean±SEM of percentage of anergic CD73^{hi}FR4^{hi} cells among CD4⁺Foxp3⁻CD62L⁻CD44^{hi} Tem cells and percentage of Nrp-1⁺ cells among anergic CD73^{hi}FR4^{hi} Tem cells are shown for 5-10 mice in each group. ***p<0.001.

FIGS. 18A-18C show that Haplo-MC increased CD62L⁻Helios⁺ effector memory Tregs and Nrp-1⁺Helios⁻ pTreg cells. MNC from SPL, PancLN and pancreas of Haplo-MC NOD were analyzed at day 60 after HCT for CD62L⁻Helios⁺ effector memory Tregs and Helios⁻Nrp-1⁺ pTreg cells. FIG. 18A: Representative patterns and mean±SEM of Foxp3⁺ Treg cells among total host-type CD4⁺ T cells, n=7-13. FIG. 18B: Representative patterns and mean±SEM of percentage of CD62L⁻Helios⁺ effector memory Treg cells among total Foxp3⁺CD4⁺ Treg cells in the spleen, PancLN and pancreas (n=7-13). FIG. 18C: Representative patterns and mean±SEM of percentage of Nrp-1⁺ pTreg cells among host-type Helios⁻ pTregs cells in SPL, PancLN and pancreas, n=5-10. P values were calculated using one-way ANOVA (*p<0.05, ***p<0.001, **** p<0.0001).

FIG. 19 shows that host-type Tregs in euthymic NOD mice with Haplo-MC upregulate expression of activation markers. 60 days after HCT, host-type CD45.1⁺Foxp3⁺CD4⁺ Treg cells in the spleen and pancreatic LN were analyzed for surface markers of CTLA4, ICOS and GITR. Representative patterns and mean±SEM of medium fluorescent intensity (MFI) of CTLA-4, ICOS and GITR expressed on host-type tTreg cells are shown for 5-11 mice in each group. *p<0.05.

FIGS. 20A-20C show that Haplo-MC in thymectomized NOD mice increased host-type CD62L⁻Helios⁺ tTreg but not CD62L⁻Helios⁻Nrp-1⁺ pTreg cells. Thymectomized NOD mice with or without induction of Haplo-MC described in FIG. 5 were further analyzed for host-type Treg subsets among residual host-type T cells from PancLN of mice with Haplo-MC, mice given conditioning alone and mice without treatment. FIG. 20A: Gated Foxp3⁺CD4⁺ Treg cell are shown in Foxp3 versus FSC. FIG. 20B: Gated Foxp⁺CD4⁺ Treg cells are shown in Helios versus CD62L. FIG. 20C: Gated Helios⁻ Treg cells are shown in Nrp-1 versus FSC. Mean±SE of percentage of Foxp3⁺CD4⁺ Treg cells among total host-type CD4⁺ T cells, Helios⁺ tTreg cells among total Treg cells, and Nrp-1⁺ pTreg cells among Helios⁻ Treg cells are shown below columns 20A, 20B, and 20C, respectively. There were 5-10 mice in each group. *p<0.05, ***p<0.001.

FIGS. 21A-21C show that Haplo-MC increased percentage of donor-type CD62L⁻ effector memory Treg cells and upregulated their CTLA4 expression. 60 days after HCT, cells from SPL, PancLN and pancreas of Haplo-MC NOD and control donor mice were analyzed for percentage of donor-type Treg cells among total donor-type CD4⁺ T cells and percentage of CD62L⁻ effector memory Treg cells among total donor-type Foxp3⁺CD4⁺ Treg cells as well as Treg cell expression of CTLA4, ICOS, and GITR. FIGS. 21A and 21B: Representative patterns and mean±SEM shows percentage of Treg cells among donor-type CD4⁺ T cells or CD62L⁻Helios⁺ effector memory Treg cells among donor-type Treg. n=6-11. FIG. 21C: Representative patterns and mean±SEM of median fluorescent intensity (MFI) of CTLA-4, ICOS and GITR expressed by donor-type Tregs in spleen and PancLN, n=4-9. P values were calculated using unpaired 2-tailed Student's t tests (*p<0.05, **p<0.01, ***p<0.001).

FIGS. 22A and 22B show that Haplo-MC in thymectomized NOD mice increased donor-type CD62L⁻Helios⁺ tTreg cells. Thymectomized NOD mice with Haplo-MC described in FIG. 5 were compared with donor mice for tTreg subsets in the PancLN. FIG. 22A: Gated Foxp3⁺CD4⁺ Treg cells are shown in Foxp3 versus FSC. FIG. 22B: Gated Foxp⁺CD4⁺ Treg cells are shown in Helios versus CD62L. Mean±SE of percentage of Foxp3⁺CD4⁺ Treg cells among total host-type CD4⁺ T cells, Helios⁺ tTreg cells among total Treg cells are shown below columns 22A and 22B, respectively. There were 5-10 mice in each group. **p<0.01, ****p<0.0001.

FIGS. 23A-23B show that Haplo-MC reduced host-type pDC percentage but upregulated their PD-L1 expression. MNC from spleen of mixed chimeras and control NOD mice were analyzed at 60 days after HCT for percentage of host-type IgM-IgD⁻CD11c⁺B220⁺PDCA1⁺ (pDCs), IgM⁻IgD⁻CD11b⁻CD11c⁺CD8⁺ (CD8⁺ DCs) and IgM-IgD⁻CD11b⁺CD11c⁺ (CD11b⁺ DCs) subsets and their expression of PD-L1. FIG. 23A: Representative pattern and mean±SEM of percentage of host-type B220⁺PDCA-1⁺ pDC, CD8⁺ DC, and CD11b⁺ DC subsets (n=8-11). FIG. 23B: Representative patterns and mean±SEM of PD-L1 expression levels on host-type B220⁺PDCA-1⁺ pDC, B220⁻CD11b⁻CD8⁺ DCs, and B220⁻CD8⁻CD11b⁺ DCs, in comparison to control mice, n=6-11. P values were calculated using one-way ANOVA (*p<0.05, **p<0.01).

FIGS. 24A-24B show that Haplo-MC in thymectomized NOD mice reduced host-type pDC without changing their PD-L1 expression. Thymectomized NOD mice with Haplo-MC described in FIG. 5 were further analyzed for DC subsets. MNC from spleen of mice with Haplo-MC and mice given conditioning alone were analyzed for percentage of host-type IgM⁻IgD⁻CD11c⁺B220⁺PDCA1⁺ pDCs and their expression of PD-L1. FIG. 24A: Representative pattern and mean±SEM of percentage of host-type B220⁺PDCA-1⁺ pDC (n=5-7). FIG. 24B: Representative patterns and mean±SEM of PD-L1 expression levels on host-type pDCs population in comparison to control mice. N=5-7. *p<0.05.

FIGS. 25A-25E show that both donor and host Tregs were required to maintain tolerance status. H-2^{g/7} Haplo-MC was induced using either donor- or host mice carrying Foxp3^{DTR}. 45-60 days after HCT, diphtheria toxin (DT) was injected to chimeric mice every 3 days for 21 days. Only Foxp3⁺ Tregs cells from Foxp3^{DTR} carrying mice can express DT receptor and would be depleted. FIG. 25A: Diagram of the HCT system that allowed specific in-vivo depletion of either donor- or host-type Treg in mixed chimeras. FIG. 25B: Efficacy of depletion of Treg cells among spleen MNC was evaluated at day 21. FIG. 25C: 3 weeks after the first injection, pancreas tissue from each group was collected to evaluate insulitis (p<0.01 when comparing no depletion to host Treg depleted or both Treg depleted, p=0.17 when comparing no depletion to donor Treg depleted). Depletion of either donor or host type Treg led to moderate insulitis. Among WT mixed chimeras, more than 90% of mice were insulitis free in all the evaluated islets, this percentage dropped to 50% and 33% in donor-type Treg depleted or host-type Treg depleted chimeric mice, respectively. One representative was shown for 6-9 mice in each group (p<0.0001 when comparing no depletion to any other group. FIGS. 25D & 25E: Representative patterns and mean±SEM of percentage of CD62L⁻CD44^{hi} Tem cells among host-type Tcon cells and percentage of CD73^{hi}FR4^{hi} anergic cells among the Tem cells in the pancLN of control Haplo-MC, Haplo-MC with depletion of donor-type Treg and Haplo-MC with depletion of host-type Treg cells, n=7-12. P values were calculated using one-way ANOVA (*p<0.05, ***p<0.001).

FIGS. 26A-26B show that effective depletion of donor- or host-type Treg cells occurred after DT injections. Mixed chimerism was induced using either donor or host mice carrying Foxp3^{DTR}. 45-60 days after HCT, diphtheria toxin (DT) was injected to chimeric mice every 3 days for 21 days. Only Foxp3⁺ Treg cells from Foxp3^{DTR} carrying mice could express DT receptor and would be depleted. FIG. 26A: Depletion of donor-type Treg cells among MNC of SPL cells of mixed chimeras with donor cells carrying Foxp3^{DTR}. Representative pattern and Mean±SEM of percentage of Foxp3⁺CD4⁺ T cells among donor-type CD4⁺ T cells are shown, n=7-8. FIG. 26B: Depletion of host-type Treg cells among MNC of SPL cells of mixed chimeras with host cells carrying Foxp3^{DTR}. Representative pattern and mean±SEM of percentage of Foxp3⁺CD4⁺ T cells among host-type CD4⁺ T cells are shown. n=7-9. ****p<0.0001.

FIGS. 27A-27D show that PD-L1 expressed on host-type hematopoietic cells was required to maintain tolerance. TCD BM cells from H-2^{g/7} F1 were mixed with TCD BM cells from either WT or PD-L1^{-/-} NOD mice and injected into lethally irradiated 11-12 weeks old WT NOD mice as shown in FIG. 27A. FIG. 27B: T1D development curve are shown for up to 60 days after HCT (n=11-18, combined from 3 replicate experiments). FIG. 27C: 45-60 days after HCT, percentage of host-type CD62L⁻CD44^{hi} Tem cells among CD4⁺ Tcon or CD8⁺ Tcon cells in the pancreatic LN (left) and pancreas (right) were measured. Representative patterns and mean±SEM are shown, n=6-9. FIG. 27D: Percentage of anergic CD73^{hi}FR4^{hi}CD4⁺ Tcon cells among CD62L⁻CD44^{hi}CD4⁺ Tem cells in the pancreatic LN (left) and pancreas (right), n=6-7. P values were calculated using unpaired 2-tailed Student's t tests (*p<0.05, **p<0.01).

FIG. 28 shows that mixed chimerism status was achieved in WT NOD mice by co-transplanting TCD-BM from H-2^{g/7} F1 donor and WT or PD-L1^{-/-} host NOD mice. TCD-BM from H-2^{g/7} F1 was mixed with TCD-BM from either WT or PD-L1^{-/-} NOD mice and injected into lethally irradiated WT NOD recipients. Recipients were monitored for chimerism in blood and levels of blood glucose. Six weeks after HCT, 5 mice from each group were used for validation of Haplo-MC by analyzing mixed chimerism status of T, B and macrophage/granulocytes in the spleen and BM. Representative staining patters are shown.

FIGS. 29A-29D show the percentage and surface receptor changes of donor- or host-type Treg cells after depletion of host- or donor-type Treg cells. 3 weeks after depletion of Treg cells by DT injection as described in FIG. 25, percentage and surface receptors of donor- or host-type Treg cells in the spleen and Panc LN of NOD mice with H-2^{g/7} Haplo-MC were measured. FIGS. 29A & 29B: Representative pattern and mean±SEM of percentage of host-type Treg among host-type CD4⁺ Tcon cells as well as expression levels of CTLA-4, ICOS, GITR on host-type Tregs in the spleen and PancLN of Haplo-MC NOD with or without depletion of donor-type Treg depletion, n=6-9. FIGS. 29C & 29D: Representative pattern and mean±SEM of percentage of donor-type Treg among donor-type CD4⁺ Tcon cells as well as expression levels of CTLA-4, ICOS, GITR on donor-type Treg cells in spleen and PancLN of Haplo-MC NOD mice with or without depletion of host-type Treg depletion, n=6-9. P values were calculated using unpaired 2-tailed Student's t tests (*p<0.05, **p<0.01).

FIGS. 30A-30E show the interactions among donor- and host-type Treg cells and PD-L1^{hi} pDCs in the periphery of Haplo-MC NOD mice. Depletion of Treg cells in Haplo-MC NOD mice was described in FIG. 25, and establishing Haplo-MC with host-type PD-L1^{-/-} hematopoietic cells was described in FIG. 27. FIGS. 30A & 30B: Host-type pDCs and their expression of PD-L1 in the spleen of Haplo-MC mice with or without depletion of donor- or host-type Treg cells were compared. Representative pattern and mean±SEM of percentage of host-type B220⁺PDCA1⁺ pDC among IgM⁻IgD⁻CD11c⁺ cells and their PD-L1 expression levels are shown, n=5-9. FIGS. 30C & 30D: Host-type CD220⁺PDCA-1⁺ pDCs, CD8⁺ DC, and CD11 b⁺ DC subsets in the spleen of Haplo-MC mice with or without hematopoietic cell PD-L1 deficiency were measured. Representative pattern and mean±SEM of DC subsets are shown, n=6-8. FIG. 30E: Percentage of Helios⁻ pTregs among host- or donor-type Tregs in the spleen, PancLN and pancreas was measured. Helios⁻Nrp-1⁺ pTreg cells among Helos⁻ pTreg cells in the pancreas were also measured. Representative patterns and mean±SEM is shown, n=6-9. P values were calculated using one-way ANOVA (30A and 30B) or unpaired 2-tailed Student's t tests (30C-30E) (**p<0.01, ***p<0.001).

FIGS. 31A-31B show that PD-L1 deficiency in host-type hematopoietic cells caused no changes in the donor- or host-type Treg cells in the mixed chimeric NOD mice. Mixed chimerism was induced by transplanting TCD-BM from either WT or PD-1^{-/-} NOD mice together with TCD-BM from H-2^{g/7} F1 donors. 60 days after HCT, percentage of donor-type (CD45.2⁺) or host-type (CD45.1⁺) Treg cells (TCRβ⁺Foxp3⁺CD4⁺) among donor- or host-type CD4⁺ T cells in the spleen, PancLN, and pancreas were measured. Representative patterns and mean±SEM of percentage of Tregs (Foxp3⁺) among host-type (31A) or donor-type (31B) CD4⁺ T cells in spleen, pancreatic LN, and pancreas are shown. N=6-9.

FIGS. 32A-32C show that expansion of antigen-specific pTreg cells in the pancreas was critical for preventing T1 D in Haplo-MC BDC2.5 NOD mice. Haplo-MC in BDC2.5 NOD mice were established with BM cells from H-2^{g/7} or H-2^{s/g7} donors as described in FIG. 10. The Haplo-MC mice and control mice given conditioning alone were monitored for T1 D development by checking blood glycose. The T1 D development curve is shown in FIG. 32A. 60 days after HCT, the mixed chimeras with or without hyperglycemia was measured for percentage of Foxp3⁺ Treg cells among host-type I-A^{g7}-HIP-2.5-tetramer⁺ autoreactive CD4⁺ T cells. FIG. 32B: Representative patterns of Tetramer⁺Foxp3⁺CD4⁺ T cells. FIG. 32C: Mean±SEM of percentage of Foxp3⁺ Treg cells among I-A^{g7}-HIP-2.5-tetramer⁺ autoreactive CD4⁺ T cells. There were 4-8 mice in each group. **p<0.01, ****p<0.0001.

### DETAILED DESCRIPTION

Disclosed herein is a method of treating or preventing an autoimmune disease such as type 1 diabetes, lupus (e.g., systemic lupus erythematosus), and multiple sclerosis by inducing haplo-identical mixed chimerism in a subject. The method entails administration of non-myeloablative low doses of CY, PT, and ATG, and infusion of CD4+T-depleted hematopoietic transplant from a donor, to the subject who suffers from an autoimmune disease.

The terms “treat,” “treating,” or “treatment” as used herein with regards to a condition refers to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

The term “low dose” as used herein refers to a dose of a particular agent, such as cyclophosphamide (CY), pentostatin (PT), or anti-thymocyte globulin (ATG), and is lower than a conventional dose of each agent used in a conditioning regimen, particularly in a myeloablative conditioning regimen. For example, the dose may be about 5%, about 10%, about 15%, about 20% or about 30% lower than the standard dose for conditioning. In certain embodiments, a low dose of CY may be from about 30 mg/kg to about 75 mg/kg; a low dose of PT is about 1 mg/kg; and a low dose of ATG may be from about 25 mg/kg to about 50 mg/kg. In general, different animals require different doses and human doses are much lower than mouse doses. For example, a low dose for BALB/c mice is about 30 mg/kg, for C57BL/6 mice is from about 50 mg/kg to about 75 mg/kg or from about 50 mg/kg to about 100 mg/kg, and for NOD mice is about 40 mg/kg.

In some embodiments, the human dose of CY used in the conditioning regimens and methods described herein may be from about 50 mg to about 1000 mg, from about 100 mg to about 800 mg, from about 150 mg to about 750 mg, from about 200 mg to about 500 mg, about 100 mg, about 200 mg, about 300 mg, about 400 mg, about 500 mg, about 600 mg, about 700 mg, or about 800 mg. In some embodiments, the human dose of ATG used in the conditioning regimens and methods described herein may be from about 0.5 mg/kg/day to about 10 mg/kg/day, from about 1.0 mg/kg/day to about 8.0 mg/kg/day, from about 1.5 mg/kg/day to about 7.5 mg/kg/day, from about 2.0 mg/kg/day to about 5.0 mg/kg/day, about 0.5 mg/kg/day, about 1.0 mg/kg/day, about 1.5 mg/kg/day, about 2.0 mg/kg/day, about 2.5 mg/kg/day, about 3.0 mg/kg/day, about 3.5 mg/kg/day, about 4.0 mg/kg/day, about 4.5 mg/kg/day, or about 5.0 mg/kg/day. In some embodiments, the human dose of PT used in the conditioning regimens and methods described herein may be from about 1 mg/m²/dose to about 10 mg/m²/dose, from about 2 mg/m²/dose to about 8 mg/m²/dose, from about 3 mg/m²/dose to about 5 mg/m²/dose, about 1 mg/m²/dose, about 2 mg/m²/dose, about 3 mg/m²/dose, about 4 mg/m²/dose, about 5 mg/m²/dose, about 6 mg/m²/dose, about 7 mg/m²/dose, about 8 mg/m²/dose, about 9 mg/m²/dose, or about 10 mg/m²/dose.

In another aspect, the conditioning regimens and methods described herein include administering the CY, PT, and/or ATG on a daily, weekly, or other regular schedule. For example, administration of CY may be daily; administration of PT may be weekly or at an interval greater than every day (e.g., every two, every three, or every four days); and administration of ATG may be daily, weekly, or at an interval greater than every day (e.g., every two or three days).

In certain embodiments, a dose of CY may be administered to the recipient on a daily basis for up to about 28 days, up to about 21 days, up to about 14 days, up to about 12 days or up to about 7 days prior to transplantation. In certain embodiments, a dose of CY may be administered to the recipient every other day for up to about 28 days, up to about 21 days, up to about 14 days, or up to about 7 days prior to transplantation. In one example, a dose of CY may be administered to the recipient on a daily basis for about 21 days prior to transplantation.

In certain embodiments, a dose of PT may be administered to the recipient every day, every other day, every third day, every fourth day, every fifth day, every sixth day, or every week for up to about 28 days, up to about 21 days, up to about 14 days, up to about 12 days or up to about 7 days prior to transplantation. In one example, a dose of PT may be administered to the recipient every week for about 21 days prior to transplantation. In another example, a dose of PT may be administered to the recipient every two, three, or four days starting about 3 weeks prior to transplantation. In yet another example, 3 doses of PT may be administered to the recipient for a week starting about 3 weeks prior to transplantation.

In certain embodiments, a dose of ATG may be administered to the recipient every other day, every third day, every fourth day or every fifth day for up to about 28 days, up to about 21 days, up to about 14 days, up to about 12 days or up to about 7 days prior to transplantation. For example, a dose of ATG may be administered to the recipient every third day for about 21 days prior to transplantation. In certain embodiments, a dose of ATG may be administered for two, three, or four days in a row about 7 days, 8 days, 9 days, 10 days, 11 days, 12 days, 13 days, or 14 days prior to transplantation. In certain embodiments, a dose of ATG may be administered for 5 days in a row starting about two weeks prior to transplantation.

In one embodiment, the conditioning regimen includes (i) three doses of PT at a dose of about 4 mg/m²/dose may be administered to a human patient about 3 weeks, about 2 weeks and about 1 week before transplantation; (ii) three, four, or five doses of ATG at a dose of about 1.5 mg/kg/day may be administered to a human patient about 12 days, about 11 days, and about 10 days before transplantation; and (iii) CY at a dose of about 200 mg orally may be administered to a human patient on a daily basis about 3 weeks before transplantation.

It is within the purview of one of ordinary skill in the art to select a suitable route of administration of CY, PT and ATG. For example, these agents can be administered by oral administration including sublingual and buccal administration, and parenteral administration including intravenous administration, intramuscular administration, and subcutaneous administration. In a preferred embodiment, one or more of CY, PT and ATG are administered intravenously. In some embodiments, CY is administered orally and ATG and PT are administered intravenously.

The essential pathogenesis of autoimmune diseases (i.e. T1 D, and lupus) lies in the abnormalities of the hematopoietic stem cells (HSC) (9, 10) because an autoimmune disease can be transferred from potential autoimmune patients into non-autoimmune patients via HLA-matched allogeneic HCT (11). The abnormalities of hematopoietic stem cells can lead to development of defective central and peripheral immune tolerance mechanisms that allow development of systemic or organ-specific autoimmune diseases including T1 D, systemic lupus erythematosus (SLE), and multiple sclerosis (MS) (12).

NOD mouse model has provided invaluable understanding of basic immune pathogenesis, genetic and environmental risk factors, and immune targeting strategies (13, 14). HSC from NOD mice give rise to thymic medullary DCs that express I-A^{g7} that cannot mediate effective negative selection of autoreactive T cells or effective production of thymic Treg (tTreg) cells, leading to defective function of tTreg cells and loss of tolerogenic features of dendritic cells in the periphery (15, 16) including tolerogenic PD-L1^{hi} plasmacytoid dendritic cells (pDCs) becoming non-tolerogenic PD-L1^{lo} pDCs. Owing to these defects, co-stimulatory blockade could not induce transplantation immune tolerance in NOD mice (17).

Previous publications with murine models have demonstrated that induction of full MHC-mismatched mixed chimerism cures established autoimmune diseases such as T1 D, systemic lupus, and MS without causing graft versus host disease (GVHD) (18-22). Unfortunately, full HLA-mismatched HCT is not yet applicable in clinic. Therefore, induction of Haplo-MC to reverse established autoimmunity in T1 D mice is tested in this disclosure, with a non-myeloablative conditioning regimen of anti-thymocyte globulin (ATG)+cyclophosphamide (CY)+pentostatin (PT), and induction of donor CD4⁺ T-depleted hematopoietic transplant. As demonstrated in the working examples, induction of Haplo-MC cured established T1 D in both euthymic and adult-thymectomized NOD mice, with re-establishing both central and peripheral tolerance.

Autoimmune T1D is associated with particular MHC (HLA) in mouse and humans (53, 54) and arises from defects in both central and peripheral tolerance mechanisms (55). It was previously reported that induction of full MHC-mismatched but not MHC-matched mixed chimerism was able to reverse autoimmunity in prediabetic, new-onset and late-stage diabetic WT NOD mice(18-20); full MHC-mismatched but not matched mixed chimerism augmented thymic negative selection of autoreactive T cells and tolerized residual autoreactive T cells in the periphery of BDC2.5 NOD mice with transgenic autoreactive T cells(6, 51). However, full-MHC-mismatched mixed chimerism is not yet applicable in clinic. Although haploidentical HCT is now widely used in clinic (1), whether haplo-identical mixed chimerism (Haplo-MC) could cure autoimmunity remains unknown, because MHC (HLA)-matched mixed chimerism cannot reverse autoimmunity in mice or humans (6, 7). Although full MHC-mismatched mixed chimerism can reverse autoimmunity in WT NOD mice and augment thymic negative selection and peripheral tolerance of autoreactive T cells in transgenic BDC2.5 NOD mice, the cellular mechanisms of tolerance and how thymic Treg cells regulate peripheral DCs and pTreg cells in the mixed chimera remains unclear.

As demonstrated herein, with conditioning regimen of ATG+CY+PT and depletion of CD4⁺ T cells in transplant, induction of Haplo-MC effectively cures the established autoimmunity with elimination of insulitis in both euthymic and adult-thymectomized NOD mice, with not only H-2^{g/7} F1 donors that possess autoimmune resistant H-2^{b} but also H-2^{s/g7} donors that possess autoimmune susceptible H-2^{s}. The cure of autoimmunity in thymectomized NOD mice is associated with expansion of donor- and host-type Treg cells and anergy of residual host-type T cells. The cure of autoimmunity in euthymic NOD mice is associated with preferential augmentation of negative selection of host-type autoreactive thymocytes and generation of tTreg cells in the thymus, as well as associated with expansion of activated tTreg cells, upregulation of pDC expression of PD-L1, and preferential expansion of host-type pTreg cells in the periphery. On the other hand, Haplo-MC in euthymic NOD mice established with myeloablative TBI-conditioning and infusion of TCD-BM cells from the H-2^{b/g7} or H-2^{s/g7} donors was not able to eliminate insulitis, although it prevented clinical T1 D development. These observations are novel and also support a theory proposed by Sykes and colleagues that cure of established autoimmunity by induction of mixed chimerism via allogeneic HCT requires 1) graft versus autoimmune cells (GVA) activity; 2) thymic depletion; 3) peripheral anergy and deletion of autoreactive T cells; and 4) expansion of Treg cells(12).

First, GVA activity in the absence of GVHD is important. Induction of Haplo-MC without causing GVHD in recipients conditioned with non-myeloablative ATG+CY+PT requires infusion of CD4+T-depleted hematopoietic transplant containing donor CD8+T, NK and other cells (56). And induction of Haplo-MC in recipients conditioned with myeloablative TBI requires infusion of donor TCD-BM cells (29). As disclosed herein, the former but not the latter approach was able to eliminate insulitis in Haplo-MC NOD mice, although both approaches prevented clinical T1 D development. Therefore, infusion of CD4+T-depleted hematopoietic graft containing lymphocytes such as CD8+T and NK cells that mediate GVA activity plays an important role in eliminating residual autoreactive T cells in the mixed chimeras.

Second, Haplo-MC with donors that possess autoimmune-susceptible H-2^{s} is as effective as Haplo-MC with donors that possess autoimmune-resistant H-2^{b} in augmenting negative selection and generation of tTreg cells in the thymus. As demonstrated in the working examples, both H-2^{g/7} and H-2^{s/g7} mixed chimeras showed partial depletion of host-type CD4⁺CD8⁺ (DP) thymocytes in WT NOD and near complete depletion of the DP thymocytes in BDC2.5 NOD with transgenic autoreactive CD4⁺ T cells. In contrast, there was a marked expansion of host-type tTreg cells among CD4⁺CD8⁻ thymocytes in both WT and BDC2.5 NOD mice with H-2^{b/g7} and H-2^{s/g7} chimerism. Based on the partial deletion of DP thymocytes in the thymus of WT NOD and complete deletion of DP thymocytes in the thymus of BDC2.5 NOD with transgenic autoreactive T cells, induction of Haplo-MC preferentially augments thymic negative selection of autoreactive T cells, with augmentation of tTreg generation in NOD mice.

Surprisingly, autoimmune susceptible H-2^{s} is as effective as autoimmune-resistant H-2^{b} in augmenting negative selection and expansion of host-type Treg cells in the Haplo-MC NOD mice, despite being unable to augment negative selection or prevent T1 D development when backcrossed to NOD mice (23). This may result from different H-2^{s} cell distribution in H-2^{s/g7} Haplo-MC NOD mice and H-2^{s/g7} NOD mice. When H-2^{s} is backcrossed to NOD mice, H-2^{s} is expressed by both thymic cortical and medullar epithelial cells and DC cells. In this case, similar to I-A^{g7}, I-A^{s} is involved in both positive and negative selection, and manifests with defective negative selection (23). However, in the H-2^{g7/s} Haplo-MC, cortical epithelial cells express I-A^{g7} without I-A^{s}. Donor-type DCs that express I-A^{g7/s} are present in the thymic medullary. For the thymocytes positively selected by only I-A^{g7} in thymic cortex, MHCII of I-A^{s} expressed by donor-type DCs in the medullary is equivalent to an “allo-MHC.” TCRs have particular high binding affinity towards foreign MHC (57). The high binding affinity leads to augmentation of negative selection of host-type Tcon cells, in particular, host-type cross-reactive autoreactive Tcon cells. It was previously shown that many autoreactive T cells are cross-reactive, and MHC-mismatched mixed chimeras preferentially deplete those cross-reactive T cells (32). On the other hand, the high binding affinity leads to augmentation of Foxp3⁺ tTreg generation (58). In addition, augmented deletion of autoreactive T cells, especially the cross-reactive autoreactive T cells, may make the residual autoreactive T cells susceptible to Treg suppression in the periphery. It was reported that T cells from NOD mice or T1 D patients are resistant to Treg suppression (59).

Third, Haplo-MC preferentially augments deletion and induction of anergy of host-type T cells in the periphery of NOD mice. As demonstrated in the working examples, elimination of insulitis in euthymic and thymectomized WT NOD mice was associated with marked reduction in yield although not in percentage of CD44^{hi}CD62L⁻ effector memory host-type T cells in the pancreatic LN and pancreas as well as an increase in the percentage of CD73^{hi}FR4^{hi} anergic cells among residual host-type I cells. Haplo-MC in the euthymic NOD mice completely deleted autoantigen-specific HIP-2.5-tetramer⁺CD4⁺ and NRP-V7-tetramer⁺CD8⁺ T cells among host-type T cells in the pancreas. Therefore, Haplo-MC can preferentially mediate deletion and anergy of host-type autoreactive T cells in the peripheral lymphoid tissues and autoimmune target organs.

Fourth, cure of autoimmunity with elimination of insulitis in euthymic and thymectomized Haplo-MC NOD mice is associated with differential expansion of tTreg and pTreg cells. T1 D pathogenesis in NOD mice or T1 D patients is associated with quantitative and qualitative defects in Treg cells (60, 61) as well as associated with Tcon cell resistance against Treg suppression (59, 62). As demonstrated in the working examples, cure with elimination of insulitis in the euthymic Haplo-MC was associated with expansion of both donor- and host-type CD62L⁻Helios⁺ tTreg cells as well as expansion of host-type CD62L⁻Helios⁻Nrp-1⁺ pTreg cells. In contrast, the cure in thymectomized Haplo-MC mice was only associated with expansion of both donor- and host-type CD62L⁻Helios⁺ tTreg cells. Accordingly, induction of Haplo-MC allows Treg cells to suppress residual autoreactive T cells; and activation and expansion of donor- and host-type tTreg cells are sufficient for controlling residual autoreactive T cells in thymectomized Haplo-MC, but additional expansion of host-type pTreg cells is also required for controlling residual autoreactive T cells in the euthymic Haplo-MC.

Fifth, Haplo-MC in euthymic mice restores peripheral pDC tolerance status with upregulation of PD-L1 and augments pTreg expansion. It has been reported that Foxp3⁻CD73^{hi}FR4^{hi}Nrp-1⁺CD4⁺ T cells can be the precursors of Foxp3⁺ pTreg cells (41); PD-L1 interaction with PD-1 on activated Tcon cells can augment their transdifferentiation into pTreg cells (63); PD-1 signaling also stabilized Foxp3 expression in pTreg cells (64); and PD-L1 interaction with CD80 on Treg cells augmented Treg cell survival and expansion (65, 66). Consistently, Haplo-MC NOD mice showed expansion of both donor- and host-type Helios⁺CD62L⁻ effector memory tTreg and expansion of Helios⁻CD62L⁻Nrp-1⁺ pTreg cells in the spleen, pancreatic lymph nodes and pancreas. In addition, the prevention of T1 D development in BDC2.5 NOD mice was associated with expansion of antigen-specific pTreg cells. Furthermore, the expansion of Helios⁻CD62L⁻Nrp-1⁺ pTreg cells was associated with expansion of anergic Foxp3⁻CD73^{hi}FR4^{hi}Nrp-1⁺CD4⁺ T cells as well as upregulation of PD-L1 by host-type pDCs.

On the other hand, depletion of either donor- or host-type Treg cells led to a marked reduction of host-type pDCs and their down-regulation of PD-L1. In contrast, PD-L1 deficiency in host-type hematopoietic cells resulted in marked reduction of host-type pDCs and severe loss of host-type pTreg cells in the PancLN and pancreas of Haplo-MC NOD mice. Therefore, donor-type and host-type tTreg cells from the thymus of Haplo-MC can restore the tolerance status of host-type peripheral pDCs by upregulating expression of PD-L1, and the PD-L1 interaction with PD-1 and CD80 on host-type autoreactive Tcon cells augments their trans-differentiation and expansion of antigen-specific pTreg cells.

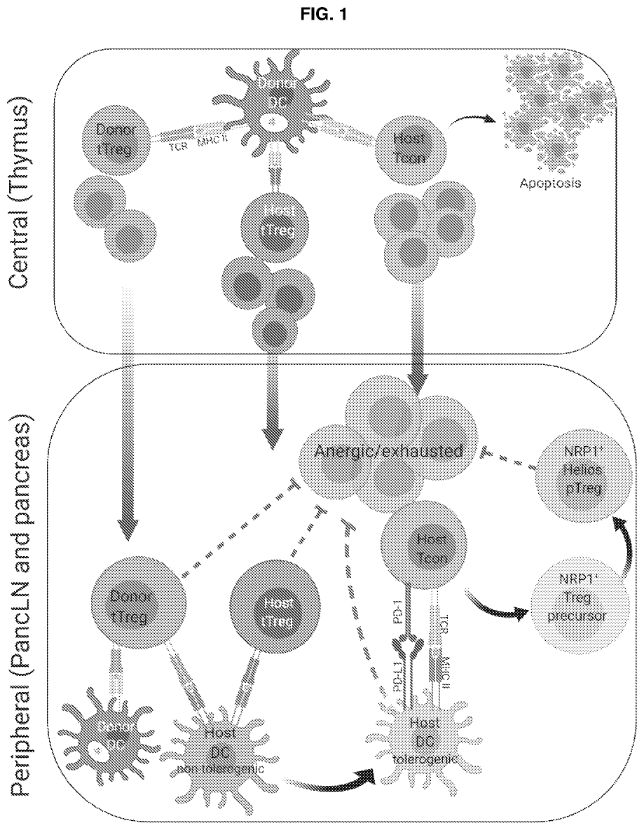

Accordingly, disclosed herein is a systemic network of allo-MHC-expressing DCs, Treg cells and tolerogenic DCs in the Haplo-MC NOD mice. As depicted in FIG. 1, induction of Haplo-MC allows allo-MHC expressing donor-type DC subsets to engraft in the host-thymus, resulting in augmentation of negative selection of host-type autoreactive T cells and production of donor- and host-type tTreg cells. The tTreg cells are activated in the periphery and restore the tolerogenic features of host-type DCs (i.e. pDCs), including upregulation of their expression of PD-L1. The interactions between tolerogenic pDCs and residual autoreactive T cells via co-inhibitory receptors such as PD-L1 interaction with PD-1 augment autoreactive T cells become anergic/exhausted T cells or become antigen-specific pTreg cells. Furthermore, the Haplo-MC is a relatively stable system. Depleting either donor-type or host-type Treg cells only causes moderate and self-limiting recurrence of insulitis in the absence of clinical T1 D; because depletion of donor-type Treg cells can lead to compensatory expansion of host-type Treg cells, or vice versa. Therefore, induction of Haplo-MC can restore both central and peripheral tolerance in T1 D mice.

As demonstrated herein, induction of Haplo-MC using non-myeloablative conditioning of ATG+CY+PT and infusion of CD4⁺ T-depleted hematopoietic transplant may have strong clinical potential as a curative therapy for refractory autoimmune diseases. First, induction of haplo-MC is more effective than matched-MC in reversal of autoimmunity. Induction of MHC (HLA)-matched mixed chimerism has been successfully achieved in humans for providing kidney transplantation immune tolerance (7, 67). However, induction of MHC(HLA)-matched mixed chimerism has been reported to not prevent lupus flare in patients (7) and to not prevent T1 D in mouse models (6). The current studies showed that induction of haploidentical mixed chimerism effectively “cure” T1 D in both euthymic and thymectomized T1 D mice, even with a donor that possesses an autoimmune susceptible MHC.

Second, the current regimen of induction of haplo-MC is likely to be applicable in clinic. Haploidentical HCT has been widely used in clinic for treating non-malignant hereditary hematological disorders (1). The current protocol for induction of Haplo-MC with conditioning regimen of ATG+CY+PT and infusion of donor CD4⁺ T-depleted transplant is now under phase I safety clinical trial with sickle cell patients (NCT03249831) and encouraging results have been obtained. Trials have been carried out with two sickle cell patients. Although no detectable chimerism in the first patient was achieved, when CY dose during conditioning was increased, the second patient reached 180 days after HCT and developed mixed chimerism for CD34⁺ stem cells in the bone marrow as well as mixed chimerism for T, B, NK and myeloid cells in the peripheral blood. The patient has predominantly donor-type healthy Hb with little Hbs and has total disappearance of clinical manifestation of sickle cell anemia with total absence of GVHD (data not shown).

Third, depletion of donor CD4⁺ T cells in the hematopoietic transplant may be critical for induction of stable haplo-identical mixed chimerism. Stable haploidentical mixed chimerism is currently difficult to achieve in humans (4, 5, 68). However, induction of stable Haplo-MC in humans may be achievable with conditioning regimen of ATG+CY+PT and infusion of CD4⁺ T-depleted hematopoietic transplant, and the depletion of donor CD4⁺ T cells may be critical. It was reported that depletion of CD4⁺ T cells allowed tissue-PD-L1 to tolerize infiltrating CD8⁺ T cells (25). It was necessary to use CD4⁺ T-depleted donor-spleen cells to induce stable mixed chimerism in mice (56). Recent studies also showed that adding back donor CD4⁺ T cells to transplants led to either graft rejection when low dose of bone marrow transplant was used or led to complete chimerism when high dose of donor bone marrow transplant was used; and that the presence of donor CD4⁺ T cells markedly reduced donor- and host-type T tolerance after HCT (data not shown). Thus, depletion of donor CD4⁺ T cells in hematopoietic transplant may promote establishing stable Haplo-MC in non-myeloablatively conditioned recipients.

Therefore, the working examples demonstrate induction of Haplo-MC with non-myeloablative conditioning regimen of ATG+CY+PT and depletion of donor CD4+ T cells in hematopoietic transplants cured established autoimmunity with elimination of insulitis in both euthymic and adult-thymectomized NOD mice. A central and peripheral tolerance network in the Haplo-MC NOD mice was revealed. These studies provide insights into the tolerance mechanisms in Haplo-MC and may help improvement of present protocols for treating patients with established autoimmune diseases. These studies have also laid a basic foundation for translating induction of Haplo-MC in clinic and for a clinical trial with autoimmune patients.

The following examples are intended to illustrate various embodiments of the invention. As such, the specific embodiments discussed are not to be constructed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### Example 1: Materials and Methods

Mice: All recipient mice were either purchased from National Cancer Institute animal production program (Frederick, Md., USA) or Jackson Laboratory (Bar Harbor, Me.) or were bred at City of Hope Animal Research Center. Detailed information of each strain is described in Table 1. All mice were housed in specific pathogen-free rooms in the City of Hope Animal Research Center.

TABLE 1
Animals

| Mouse | Source | Cited as |
|---|---|---|
| NOD/ShiLtJ | Jackson Laboratory, stock | WT NOD |
| | No: 001976 | |
| NOD.Cg- | Jackson Laboratory, stock | BDC2.5 NOD |
| Tg(TcraBDC2.5,TcrbBDC2.5)1D | No: 004460 | |
| oi/DoiJ | | |
| NOD/ShiLtJ-Tg(Foxp3- | Jackson Laboratory, stock | Foxp3^{DTR} NOD |
| HBEGF/EGFP)1Doi/J | No:028763 | |
| B6.NOD-(D17Mit21- | Jackson Laboratory, stock No: | PD-L1^{-/-} NOD |
| D17Mit10)/LtJ | 003300 | |
| NOD.B6-Cd274tm1Shr/J | Jackson Laboratory, stock No: | g7 |
| | 018307 | |
| SJL/J | Jackson Laboratory, stock No: | SJL |
| | 000686 | |
| C57BL/6 | National Cancer Institute | B6 |
| B6.129(Cg)- | Jackson Laboratory, stock No: 016958 | Foxp3^{DTR} B6 |
| Foxp3tm3(DTR/GFP)Ayr/J | | |
| (B6 × g7)F1 | backcrossing B6♀ to g7♂ | (B6 × g7)F1 or H- |
| | | 2^{b/g7} F1 |
| (SJL × g7)F1 | backcrossing SJL♀ to g7♂ | (SJL × g7)F1 or H- |
| | | 2^{s/g7} F1 |
| Foxp3^{DTR} F1 | backcrossing Foxp3^{DTR} B6♀ to g7♂ | Foxp3^{DTR} F1 |

Experimental procedures and materials: Induction of mixed chimerism with Cyclophosphamide (CY)+Pentostatin (PT)+Anti-thymocyte globulin (ATG) conditioning regimen, histopathology staining and insulitis evaluation, in vivo Treg depletion, induction of host lymphocyte PD-L1−/− mixed chimerism, isolation of lymphocytes from pancreas, release of dendritic cells from spleen, flow cytometry analysis including tetramer staining and detailed antibody information are disclosed below.

Induction of mixed chimerism with CY+PT+ATG condition regimen: Recipient mice were given I.P. injection of cyclophosphamide (Cy, 50 mg/kg for WT NOD, 40 mg/kg for BDC 2.5 NOD, purchased from Sigma-Aldrich) daily from D-12 to D-1, pentostatin (PT, 1 mg/kg, purchased from Sigma-Aldrich) on D-12, D-9, D-6, and D-3, and anti-thymocyte globulin (ATG, 25 mg/kg, purchased from Accurate Chemical & Scientific Corporation) on D-12, D-9, and D-6. On the day of HCT (DO), recipients were injected intravenously with bone marrow (BM) and spleen (SPL) cells from donor mice mixed with 500 ug purified depleting anti-mouse CD4 mAb (clone GK1.5, purchased from BioXcell). 6 weeks later, peripheral blood was collected from mice received HCT after conditioning or control mice received conditioning only and analyzed by flowcytometry.

Histopathology staining and insulitis evaluation: Pancreas was fixed in 10% formalin solution and embedded in paraffin blocks. Two slides were made for each level, and 3 different levels were sectioned for each sample. The distance between each level was 75 microns, and a total of 6 slides from each sample were cut and stained with H&E. The number of islets with insulitis, peri-insulitis or insulitis-free in all 6 slides were counted, and then the percentage of each severity level among all islets from this mouse were calculated.

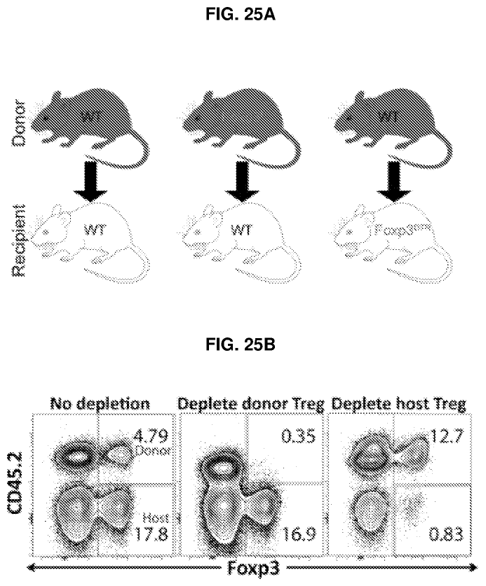

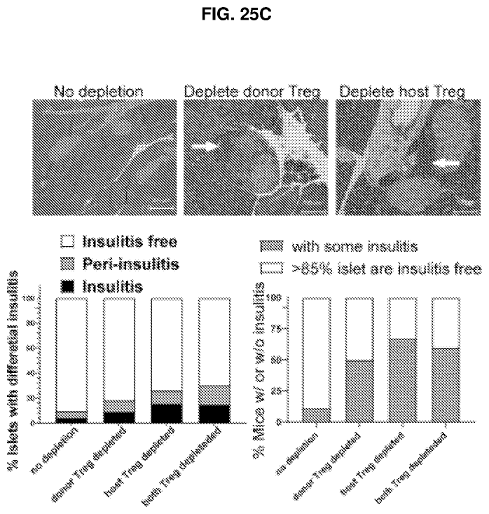

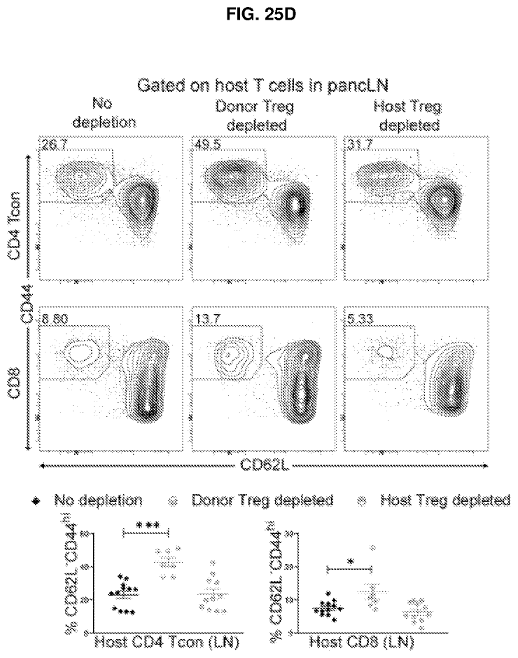

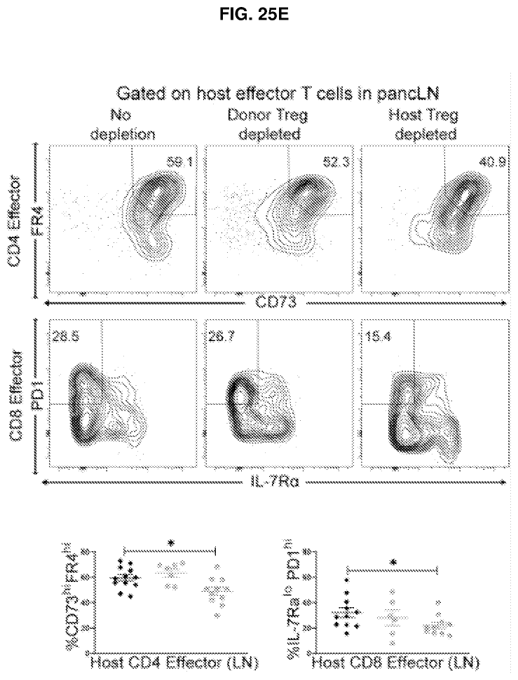

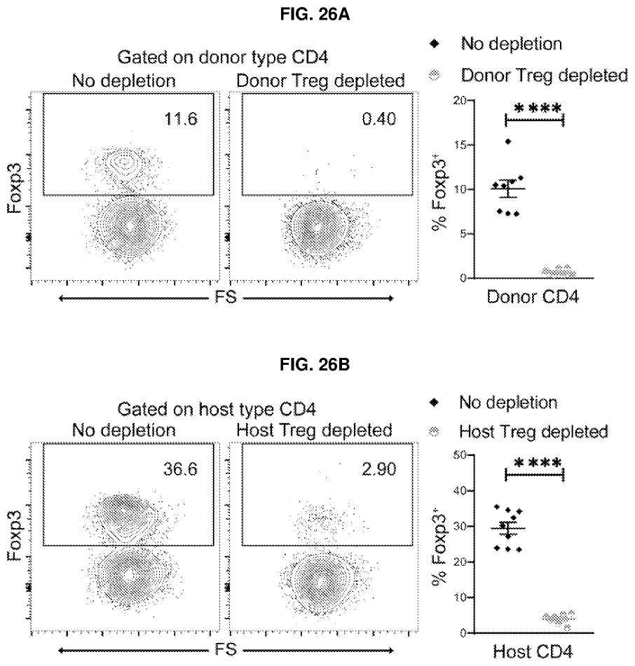

In vivo Treg depletion: A mouse model to allow donor or host specific Treg depletion was set up as illustrated in FIG. 25A, using mice listed in Table 1 in which diphtheria toxin (DT, purchased from Sigma-Aldrich) can be used to specifically ablate Foxp3+ T cells. 45-60 days after HCT, 40 ug/kg DT was injected to mixed chimeric mice intraperitoneally every 3 days for 21 days. The last two injections on day 16 and day 19 were reduced to 20 ug/kg if body weight decreased by more than 20%.

Induction of host lymphocyte PD-L1−/− mixed chimerism: Recipients were given 950 cGy total body irradiation (TBI). A cell suspension consisting of T cell depleted (TCD) BM from (B6×g7) F1 mice (7.5×10⁶) and TCD BM from WT NOD or PD-L1^{-/-} NOD mice (5×10⁶) was injected through the tail vein 8-10 hours after irradiation.

Isolation of lymphocytes from pancreas: Pancreas was kept in FACs buffer (PBS containing 2 mM EDTA and 2% BSA) on ice after harvest. It was minced quickly with a small curved scissors and mashed through a 70 um strainer. Cell suspension was centrifuged and re-suspended in 6 ml of 35% Percoll (Sigma-Aldrich, Cat #P1644-1 L) solution for each pancreas, carefully laid above 3 ml of 70% Percoll solution, centrifuged at 1200 g at room temperature for 25 minutes. After centrifuging, cells were collected from the middle layer, washed with FACs buffer, and then stained with surface antibody or tetramer antibody for flowcytometry analysis.

Release dendritic cells from spleen: Spleen was harvested and kept in cold PBS. 5 ml digestion buffer (RPMI containing 10% fetal bovine serum, collagenase D (0.15 U/ml), and DNase I (0.2 mg/ml)) was carefully injected into each spleen. Specimens were placed on an orbital shaker (80 rpm) and incubated at 37° C. for 50 minutes. After digestion, tissue was mashed through a 70 μm cell strainer and washed with FACs buffer.

Flowcytometry staining: Surface markers were stained at 4° C. for 15-20 minutes following the incubation with CD16/32 (BioXcell, Cat #. BE0307) and aqua viability dye (Invitrogen, Cat #. L34957). All intracellular staining including Foxp3, Helios and CTLA-4 were performed with the Foxp3/Transcription Factor Staining Buffer Set (eBioscience, Cat #. 00-5523-00) after surface staining. Detailed antibody information is listed in Table 2. Flowcytometry analyses were performed with a CyAn ADP Analyzer (Beckman Coulter) or LSRFortessa (BD Bioscience).

TABLE 2

| Antibodies | | | | |
|---|---|---|---|---|
| Antigen | Clone | Conjugation | Manufacture | Cat # |
| B220 | RA3-6B2 | APC- | eBioscience | 47-0452- |
| | | eFlour780 | | 82 |
| CD45.1 | A20 | APC- | eBioscience | 47-0453- |
| | | eFlour780 | | 82 |
| CD45.2 | 104 | APC- | eBioscience | 47-0454- |
| | | eFlour780 | | 82 |
| CD62L | MEL-14 | APC- | eBioscience | 47-0621- |
| | | eFlour780 | | 82 |
| CD4 | RM4-5 | APC/Fire750 | Biolegend | 109246 |
| TCR | H57-597 | APC/Fire750 | Biolegend | 100568 |
| CD45.1 | A20 | APC | eBioscience | 17-0453- |
| | | | | 82 |
| CD45.2 | 104 | APC | eBioscience | 17-0454- |
| | | | | 82 |
| Foxp3 | FJK-16s | APC | eBioscience | 17-5773- |
| | | | | 82 |
| Helios | 22F6 | APC | eBioscience | 17-9883- |
| | | | | 42 |
| CD317 (PDCA-1) | eBio927 | APC | eBioscience | 17-3172- |
| | | | | 82 |
| TCRß | H57-597 | APC | eBioscience | 17-5961- |
| | | | | 83 |
| CD304 | 3DS304M | APC | eBioscience | 17-3041- |
| (Neuropilin-1) | | | | 82 |
| B220 | RA3-6B2 | biotin | eBioscience | 13-0452- |
| | | | | 82 |
| CD11b | M1/70 | biotin | eBioscience | 13-0112- |
| | | | | 82 |
| CD11c | N418 | biotin | eBioscience | 13-0114- |
| | | | | 81 |
| F4/80 | BM8 | biotin | eBioscience | 13-4801- |
| | | | | 82 |
| Ly-6G/Ly-6C | RB6-8C5 | biotin | eBioscience | 13-5931- |
| | | | | 82 |
| CD45.1 | A20 | biotin | eBioscience | 13-0453- |
| | | | | 82 |
| CD45.2 | 104 | biotin | eBioscience | 13-0454- |
| | | | | 82 |
| CD11b | M1/70 | BUV395 | BD | 563553 |
| CD8 | 53-6.7 | BUV395 | BD | 563786 |
| CD44 | IM7 | BUV395 | BD | 740215 |
| TCRß | H57-597 | BUV395 | BD | 742485 |
| B220 | RA3-6B2 | BV421 | BD | 562922 |
| FR4 | 12A5 | BV421 | BD | 744119 |
| CD8 | 53-6.7 | BV605 | BD | 563152 |
| CD357 (GITR) | DTA-1 | BV605 | BD | 740428 |
| TCRß | H57-597 | BV605 | Biolegend | 109241 |
| CD4 | H129.19 | BV711 | BD | 740684 |
| CD8 | 53-6.7 | BV711 | BD | 563046 |
| CD278 (ICOS) | 7E.17G9 | BV711 | BD | 740763 |
| TCR VB4 | KT4 | BV711 | BD | 743023 |
| CD279 (PD1) | 29F.1A12 | BV711 | Biolegend | 135231 |
| CD11c | N418 | BV711 | Biolegend | 117349 |
| TCRß | H57-597 | BV711 | Biolegend | 109243 |
| CD45.1 | A20 | eFluor 450 | eBioscience | 48-0453- |
| | | | | 82 |
| CD45.2 | 104 | eFluor 450 | eBioscience | 48-0454- |
| | | | | 82 |
| B220 | RA3-6B2 | eFluor 450 | eBioscience | 48-0452- |
| | | | | 82 |
| CD11b | M1/70 | eFluor 450 | eBioscience | 48-0112- |
| | | | | 82 |
| Foxp3 | FJK-16s | eFluor 450 | eBioscience | 48-5773- |
| | | | | 82 |
| Helios | 22F6 | eFluor 450 | eBioscience | 48-9883- |
| | | | | 42 |
| B220 | RA3-6B2 | FITC | BD | 553088 |
| CD11c | N418 | FITC | eBioscience | 11-0114- |
| | | | | 85 |
| CD4 | GK 1.5 | FITC | eBioscience | 11-0041- |
| | | | | 82 |
| CD45.1 | A20 | FITC | BD | 553775 |
| CD45.2 | 104 | FITC | BD | 553772 |
| FR4 | eBio12A5 | FITC | eBioscience | 11-5445- |
| | | | | 80 |
| H2Kd | SF1-1.1 | FITC | BD | 553565 |
| IA/IE | M5/114.15.2 | FITC | eBioscience | 11-5321- |
| | | | | 82 |
| TCR Vß4 | KT4 | FITC | BD | 553365 |
| Foxp3 | MF23 | Alexa Fluor | BD | 560403 |
| | | 488 | | |
| CD44 | IM7 | FITC | BD | 553133 |
| TCRß | H57-597 | FITC | eBioscience | 11-5961- |
| | | | | 85 |
| CD19 | eBio1D3 | PE-cy7 | eBioscience | 25-0193- |
| | | | | 82 |
| CD3 | 145-2C11 | PE-cy7 | eBioscience | 25-0031- |
| | | | | 82 |
| CD45.1 | A20 | PE-cy7 | eBioscience | 25-0453- |
| | | | | 82 |
| CD45.2 | 104 | PE-cy7 | eBioscience | 25-0454- |
| | | | | 82 |
| CD73 | eBioTY/11.8 | PE-cy7 | eBioscience | 25-0731- |
| | (TY/11.8) | | | 82 |
| CD8 | 53-6.7 | PE-cy7 | eBioscience | 25-0081- |
| | | | | 82 |
| IgM | 11/41 | PE-cy7 | eBioscience | 25-5790- |
| | | | | 82 |
| IgD | 11-26c | PE-cy7 | eBioscience | 25-5993- |
| | | | | 82 |
| CD127 (IL-7Ra) | eBioSB/199 | PE-cy7 | eBioscience | 25-1273- |
| | (SB/199) | | | 82 |
| CD317 (PDCA-1) | eBio927 | PE-cy7 | eBioscience | 25-3172- |
| | | | | 82 |
| CD172a | P84 | PE | eBioscience | 12-1721- |
| | | | | 82 |
| CD44 | IM7 | PE | eBioscience | 12-0441- |
| | | | | 83 |
| CD8 | 53-6.7 | PE | eBioscience | 12-0081- |
| | | | | 82 |
| CD152 (CTLA-4) | UC10-4B9 | PE | eBioscience | 12-1522- |
| | | | | 82 |
| Ly-6G and Ly-6C | RB6-8C5 | PE | BD | 553128 |
| CD11b | M1/70 | PE | eBioscience | 12-0112- |
| | | | | 83 |
| CD274 (PD-L1) | MIH5 | PE | eBioscience | 12-5982- |
| | | | | 82 |
| TCR Va2 | B20.1 | PE | BD | 553289 |
| Foxp3 | NRRF-30 | PE | eBioscience | 12-4771- |
| | | | | 82 |
| CD45.2 | 104 | PerCP-Cy5.5 | eBioscience | 45-0454- |
| | | | | 82 |
| CD11b | M1/70 | PerCP-Cy5.5 | eBioscience | 101228 |
| CD73 | eBioTY/11.8 | PerCP-eFluor | eBioscience | 46-0731- |
| | (TY/11.8) | 710 | | 82 |
| CD304 | 3DS304M | PerCP-eFluor | eBioscience | 46-3041- |
| (Neuropilin-1) | | 710 | | 82 |
| CD4 | RM4-5 | Qdot605 | Invitrogen | q10092 |
| Streptavidin | N/A | Alexa Fluor | Molecular | S11249 |
| | | 350 | Probes | |
| Streptavidin | N/A | APC | eBioscience | 17-4317- |
| | | | | 82 |
| Streptavidin | N/A | APC- | eBioscience | 47-4317- |
| | | eFlour780 | | 82 |
| Streptavidin | N/A | PE-cy7 | eBioscience | 25-4317- |
| | | | | 82 |
| Armenian | eBio299Arm | PE | eBioscience | 12-4888- |
| Hamster IgG | | | | 81 |
| isotype | | | | |
| Rat lgG2a, κ | ebr2a | PE | eBioscience | 12-4321- |
| isotype | | | | 82 |
| Rat lgG2b, κ | R35-38 | BV605 | BD | 563145 |
| isotype | | | | |
| Rat IgG2b, κ | R35-38 | BV711 | BD | 563045 |
| isotype | | | | |

Tetramer staining: APC-labeled HIP 2.5 tetramer (I-A^{g7} LQTLALWSRMD), APC-labeled control tetramer (I-A^{g7} PVSKMRMATPLLMQA), PE-labeled NRP-V7 tetramer (H-2K(d) KYNKANVFL), PE-labeled control tetramer (H-2K(d) KYQAVTTTL) were obtained from the National Institutes of Health Tetramer Facility (Atlanta, Ga.). Cells were first blocked with CD16/32 for 60 minutes at 37° C., and then incubated with labeled tetramers for 90 minutes at 37° C., both CD16/32 and tetramers were diluted with complete culture media. Cells were then washed with FACs buffer and continued to regular surface marker and intracellular staining.

Statistics: Data are displayed as mean±SEM. Body weight and diabetes free rate in different groups were compared using log-rank test. Insulitis in different groups were compared using Chi-square test. Comparison of two means was done using unpaired 2-tailed Student's t test while comparison of multiple means was done using one-way ANOVA; P value of less than 0.05 is considered as significant.

Software: Flow cytometry data were analyzed with FlowJo™ Software version 10.5.3 (FlowJo LLC). Statistical analysis were prepared using GraphPad Prism software version 8.0. Abstract figure is created with BioRender.com.

Study approval: All animal procedures were approved by the IACUC of the Beckman Research Institute of City of Hope.

### Example 2: Induction of Haplo-MC Cures Autoimmunity in Established Type 1 Diabetic Euthymic NOD Mice

When autoimmune-resistant H-2^{b} were backcrossed to NOD mice, the H-2^{b/g7} NOD mice no longer developed T1 D; but when autoimmune susceptible H-2^{s} were backcrossed to NOD mice, the H-2^{s/g7} NOD mice still developed T1 D (23). Therefore, whether induction of haploidentical mixed chimerism (Haplo-MC) with H-2^{b/g7} or H-2^{s/g7} F1 donors could cure autoimmunity in both prediabetic and new-onset diabetic NOD mice was tested.

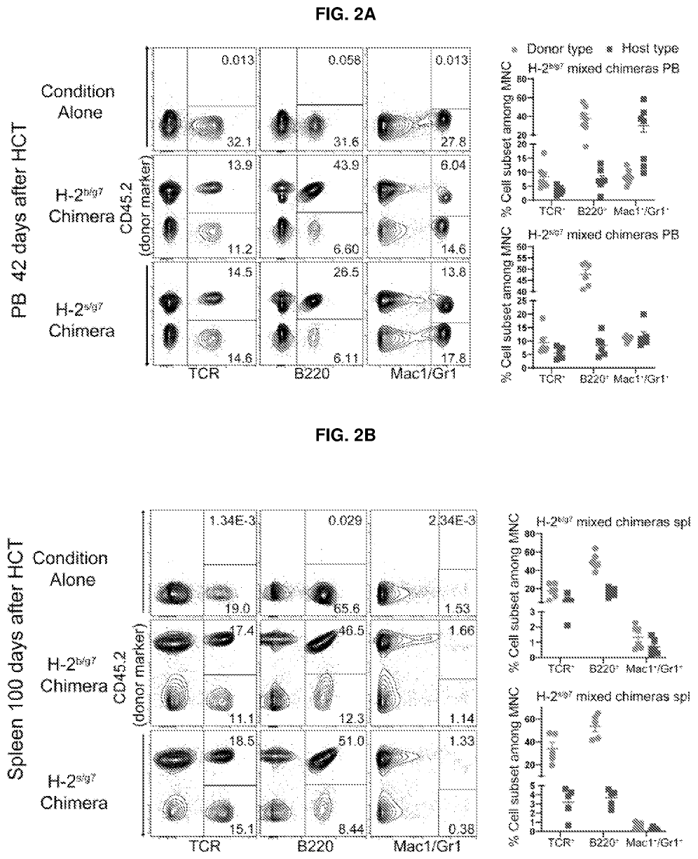

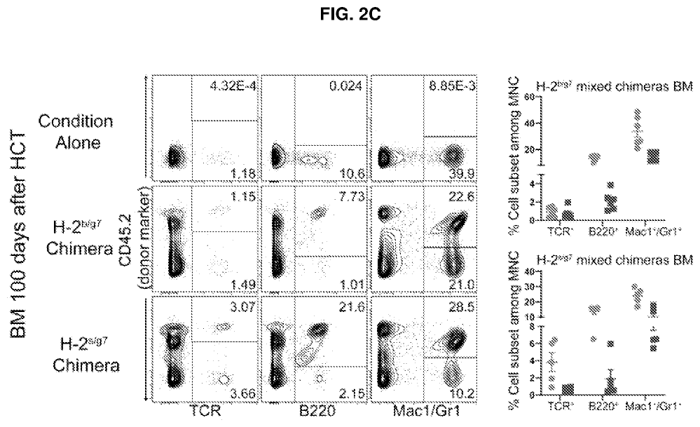

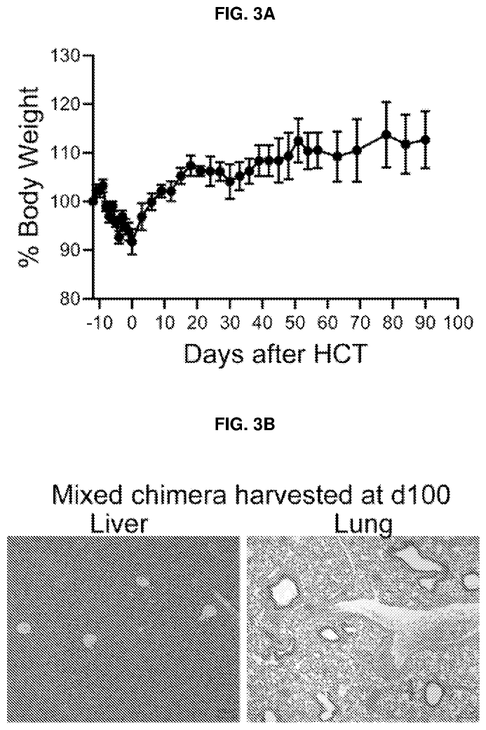

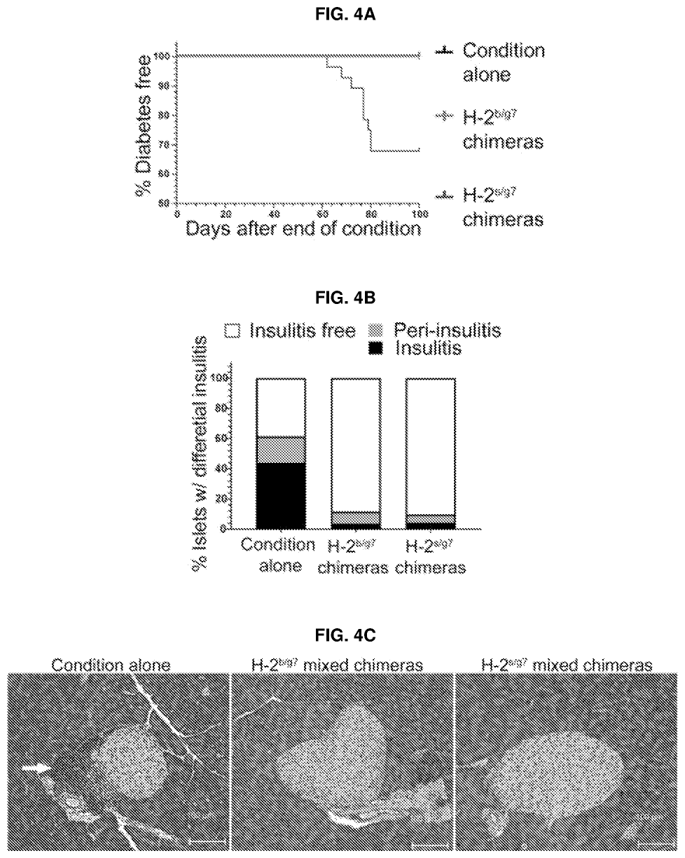

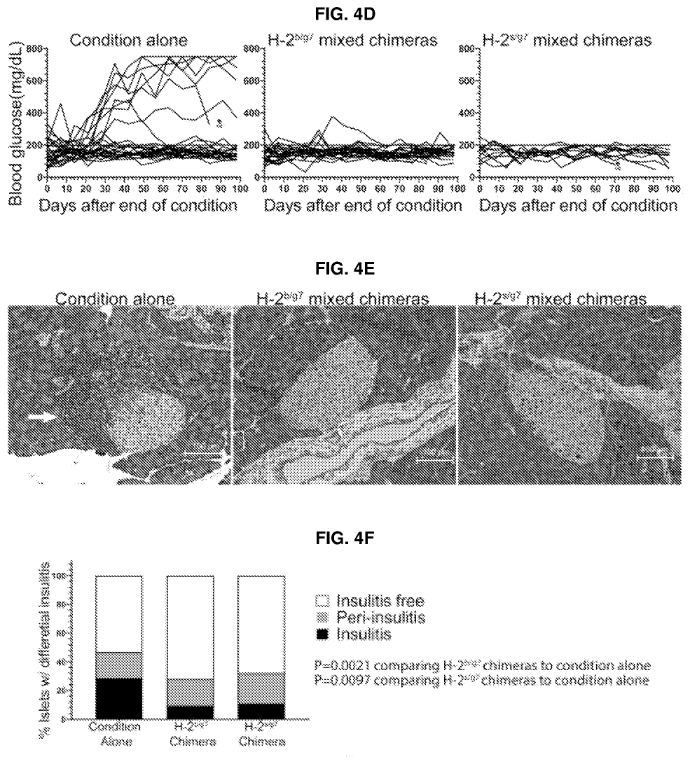

9-12 weeks old prediabetic NOD mice were conditioned with anti-thymocyte globulin (ATG)+cyclophosphamide (CY)+pentostatin (PT), as previously described (22, 24), and transplanted with bone marrow (BM, 50×10⁶) and spleen cells (30×10⁶) from H-2^{b/g7} or H-2^{s/g7} F1 donors, with co-injection of depleting anti-CD4 mAb (500 μg/mouse) to prevent acute GVHD, as previously described (25). Both haploidentical transplants resulted in stable Haplo-MC in blood, and the mixed chimerism was confirmed at the end of experiments at 100 days after HCT (FIG. 2). The mixed chimeras showed no signs of clinical GVHD as judged by their healthy appearance and stable bodyweight and no histopathological damage in GVHD target organs including liver and lung (FIG. 3). While 65% of NOD mice given conditioning alone developed hyperglycemia, and the residual mice without hyperglycemia showed severe insulitis, both recipients with H-2^{b/g7} and H-2^{s/g7} Haplo-MC showed normal glycemia for more than 100 days after HCT and showed little insulitis at the end of experiment (FIGS. 4A-4C). These results indicate that both H-2^{b/g7} and H-2^{s/g7} haplo-MC can prevent T1 D development and eliminate insulitis.

Next, Haplo-MC was induced in new-onset T1D NOD mice with blood glucose >400 mg/dL for consecutive 3 days, as previously described (20). Both H-2b/g7 and H-2s/g7 Haplo-MC normalized blood glucose with little insulitis in new-onset diabetic NOD mice (FIGS. 4D-4F). Although conditioning alone was able to normalize blood glucose in many new-onset recipients, which is consistent with previous reports (20, 26, 27), those mice still had severe insulitis (FIGS. 4D-4F).

### Example 3: Induction of Haplo-MC Cures Autoimmunity in Adult-Thymectomized NOD Mice

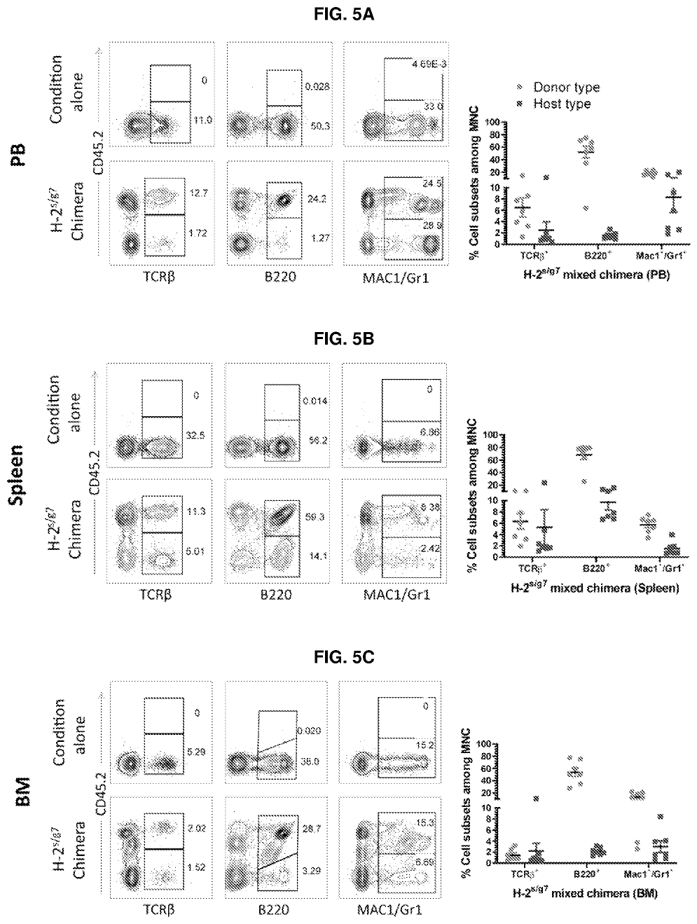

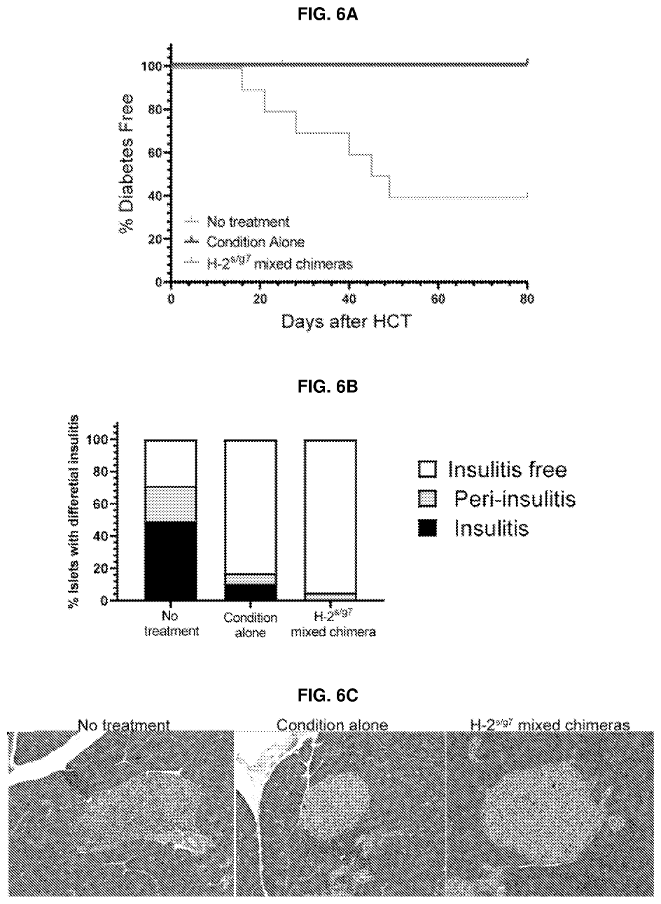

Whether functional thymus was required for preventing T1 D and eliminating insulitis was tested in Haplo-MC. Since adult (6 week old)-thymectomized NOD (Thymec-NOD) mice developed T1 D (28), whether induction of Haplo-MC in the adult Thymec-NOD mice cured T1 D was tested. Since induction of mixed chimerism with autoimmune resistant H-2^{b/g7} F1 and autoimmune susceptible H-2^{g7}/s F1 donors were equally effective at curing T1 D in NOD mice, only induction of mixed chimerism with H-2^{g7/s} F1 donors was tested in the adult Thymec-NOD mice. The same conditioning regimen of ATG+CY+PT used for euthymic NOD mice were applied to adult Thymec-NOD mice at age ˜10 weeks, that is, ˜4 weeks after thymectomy. The mice were injected with whole bone marrow (50×10⁶) from H-2^{s/g7} F1 donors. The recipients developed stable mixed chimerism as indicated by co-existence of donor- and host-type T, B, macrophage and granulocytes in the blood, spleen and bone marrow at 80 days after HCT, the end of experiments (FIGS. 5A-5C). While 60% of untreated Thymc-NOD mice developed hyperglycemia, the mice given conditioning alone or given induction of Haplo-MC didn't develop T1 D (FIG. 6A). The untreated mice with euglycemia showed severe insulitis (FIGS. 6B & 6C). Interestingly, conditioning alone markedly reduced insulitis, and induction of Haplo-MC further cleared insulitis (FIGS. 6B & 6C). These results indicate that conditioning with ATG+CY+PT alone was able to prevent T1 D development with marked reduction of insulitis in adult-thymectomized NOD mice; and induction of Haplo-MC totally eliminated residual insulitis.

### Example 4: Induction of Haplo-MC in Lethal TBI-Conditioned NOD Mice Prevents Clinical T1 D Development but is not Able to Eliminate Insulitis

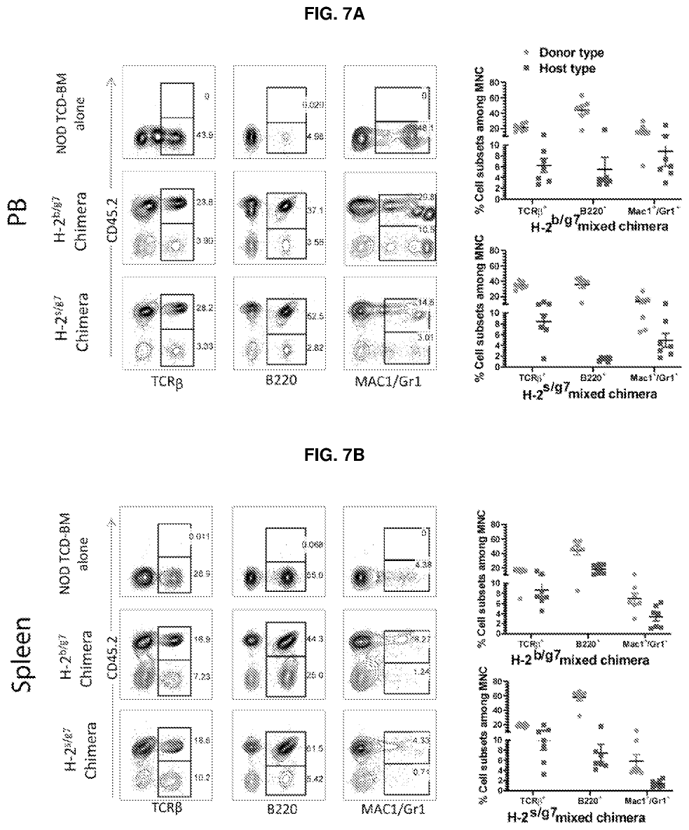

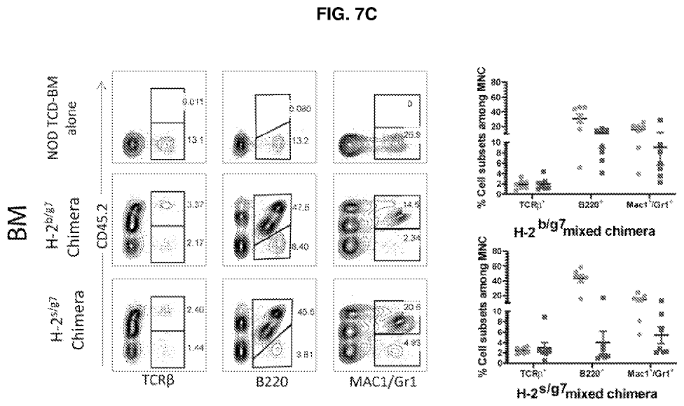

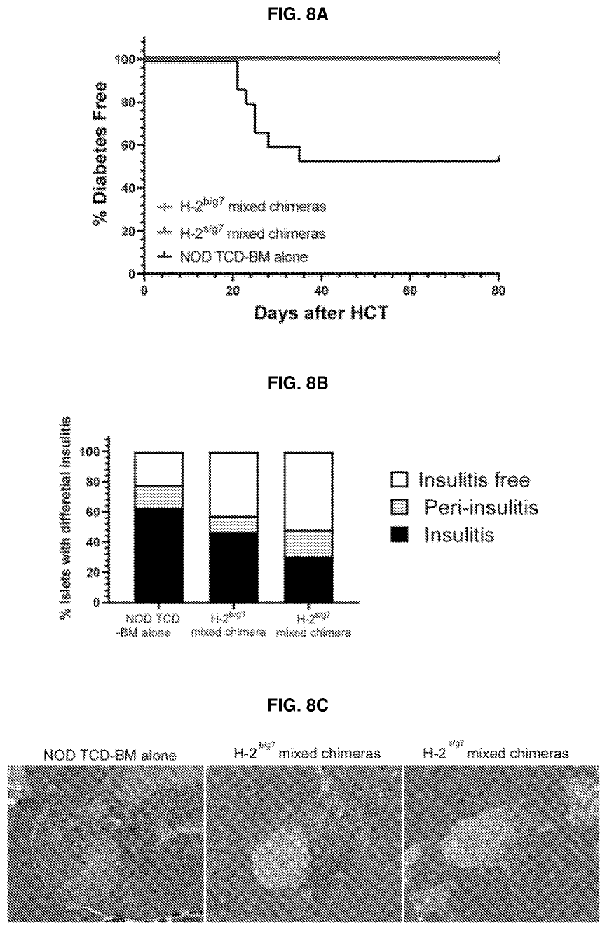

Furthermore, whether induction of Haplo-MC with myeloablative total body irradiation (950 cGy TBI) conditioning and transplantation of TCD-BM, as previously described (29), could prevent T1 D development was tested. Lethal TBI-conditioned NOD mice transplanted with syngeneic NOD TCD-BM alone (5×10⁶) were used as control. Haplo-MC was induced by transplanting TCD-BM (5×10⁶) from NOD mice and (7.5×10⁶) from H-2^{g/7} or H-2^{s/g7} F1 donors. The recipients given H-2^{b/g7} or H-2^{s/g7} TCD-BM cells developed stable mixed chimerism as indicated by co-existence of donor- and host-type T, B, macrophage and granulocytes in the peripheral blood, spleen and BM (FIG. 7). While 50% ( 7/14) of control recipients developed T1 D with hyperglycemia at ˜40 days after HCT, none of the mixed chimeras developed T1 D by 80 days after HCT (FIG. 8A). The residual control recipients with euglycemia had more than 60% of residual islets showing severe insulitis (FIGS. 8B & 8C). Surprisingly, although there was a reduction in insulitis, the mixed chimeras still had more than 30% of islets showing severe insulitis (FIGS. 8C & 8D). These results indicate that induction of mixed chimerism with TCD-BM is able to control T1 D development, but not able to eliminate insulitis.

Taken together, the above results indicate that 1) induction of Haplo-MC via non-myeloablative conditioning with CY+PT+ATG and transplantation with CD4⁺ T-depleted graft cured established T1 D with elimination of insulitis in prediabetic euthymic and adult-thymectomized as well as new-onset diabetic NOD mice; 2) induction of Haplo-MC in lethal TBI-conditioned NOD mice given donor TCD-BM cells was not able to cure T1 D autoimmunity with elimination of insulitis. In light of a theory proposed by Sykes and colleagues that graft versus autoimmune cells (GVA) activity is important for cure of autoimmunity after allogeneic HCT (12), the lack of cure in the lethal TBI-conditioned Haplo-MC NOD mice may result from transplantation of donor TCD-BM cells that have little GVH and GVA activity; and 3) the following mechanistic studies were focused on how Haplo-MC cures autoimmunity in euthymic and thymectomized NOD mice conditioned with non-myloablative regimen of ATG+CY+PT.

### Example 5: Haplo-MC in Euthymic NOD Mice Augments Thymic Negative Selection of Host-Type Thymocytes

Autoimmune NOD mice have defects in thymic negative selection (30, 31). Backcross of protective H-2^{b} but not autoimmune susceptible H-2^{s} to NOD mice was able to restore negative selection (23). The ability of Haplo-MC with H-2^{g/7} or H-2^{s/g7} donors to restore thymic deletion of host-type autoreactive T cells was tested. To avoid the confounding effects of hyperglycemia, prediabetic NOD mice having normal glycemia were used to evaluate the impact of Haplo-MC on thymocyte generation.

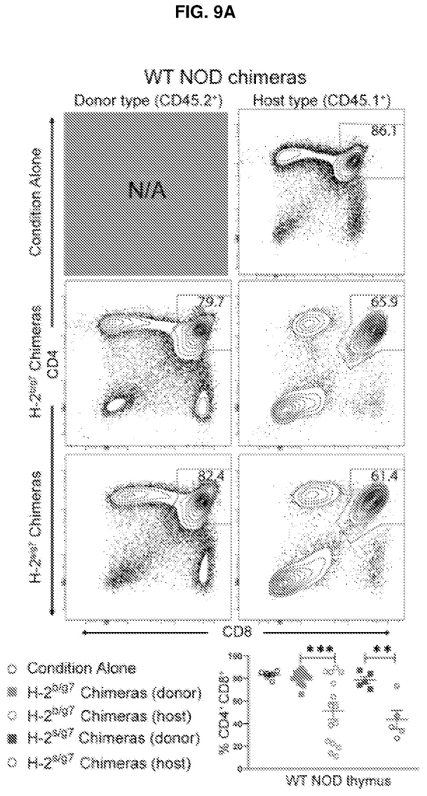

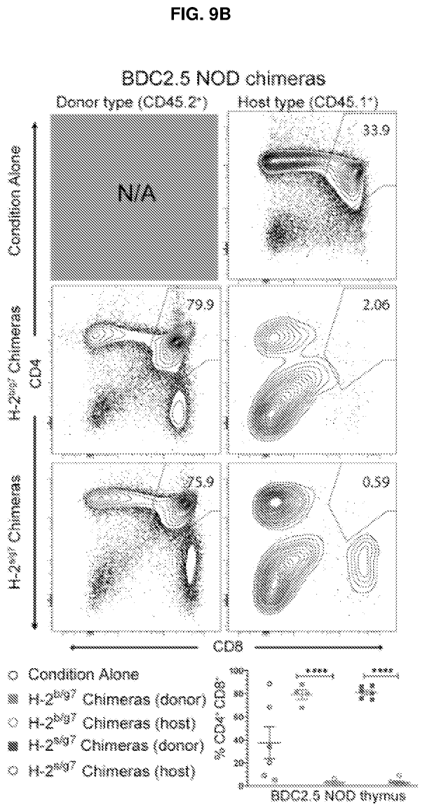

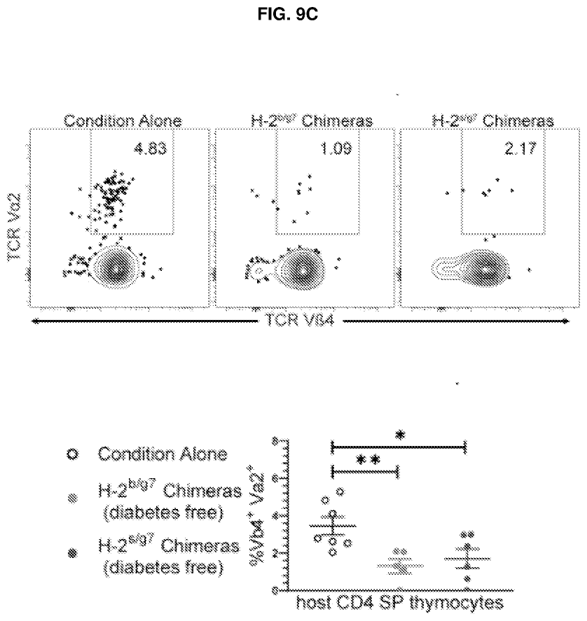

The percentage of donor-type CD4⁺CD8⁺ (DP) thymocytes in the Haplo-MC NOD mice was more than 75%, similar to that of healthy donors (FIG. 9A). This normal percentage of donor-type DP thymocytes suggest that there was no GVHD damage of thymus. The percentage of host-type DP thymocytes in the NOD mice given conditioning alone was more than 80%, however, the percentage of host-type DP thymocytes in the H-2^{b/g7} or H-2^{s/g7} Haplo-MC was significantly reduced, the average being 51.21% and 43.70%, respectively (FIG. 9A). These results suggest that haploidentical mixed chimerism with either H-2^{b/g7} or H-2^{s/g7} donors can restore negative selection in the thymus.

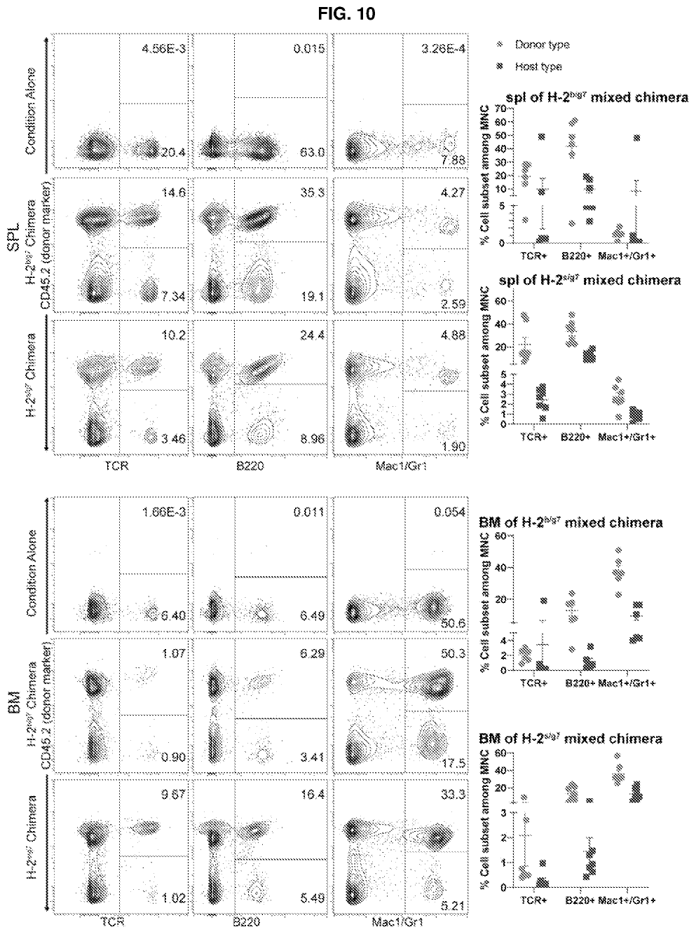

To further test whether H-2^{b/g7} or H-2^{s/g7} Haplo-MC mediated deletion of autoreactive DP thymocytes, Haplo-MC was induced in BDC2.5 NOD mice as described in FIG. 10. Both H-2^{b/g7} and H-2^{s/g7} Haplo-MC depleted almost all DP thymocytes in BDC2.5 NOD mice (FIG. 9B). In addition, autoreactive T cells often express dual TCRα (32, 33). The Vα1Vβ4 transgenic CD4⁺ T cells can express the second TCR with endogenous Vα2 (Vα2⁺Vβ4⁺)(32). As shown in FIG. 9C, the Vβ4⁺ transgenic CD4⁺ T cells with endogenous Vα2⁺ among residual CD4⁺CD8⁻ (SP) thymocytes were markedly reduced. These results indicate that induction of Haplo-MC augments negative selection of host-type thymocytes including autoreactive thymocytes.

### Example 6: Haplo-MC in Euthymic NOD Mice Augments Thymic Generation of Host- and Donor-Type Foxp3⁺ tTreg Cells

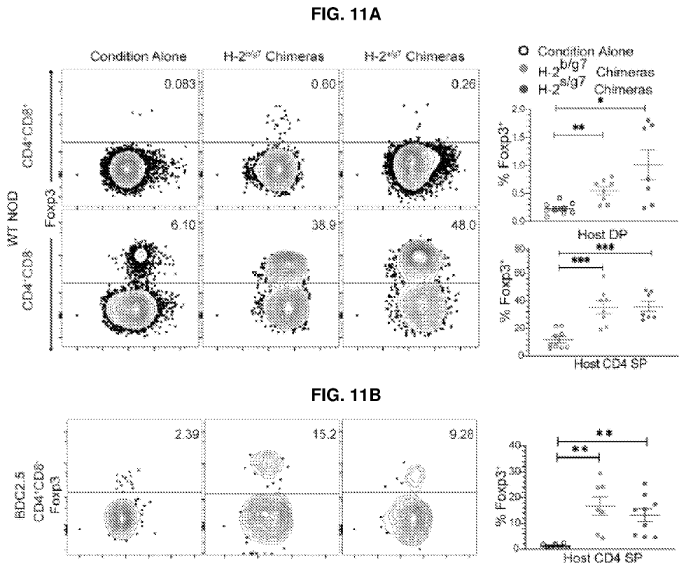

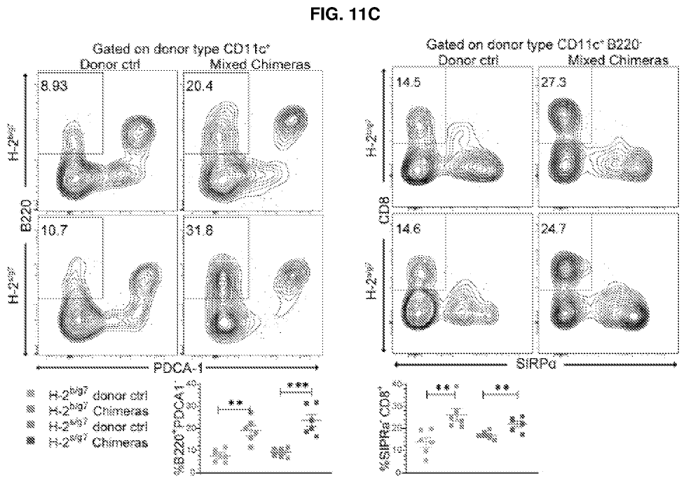

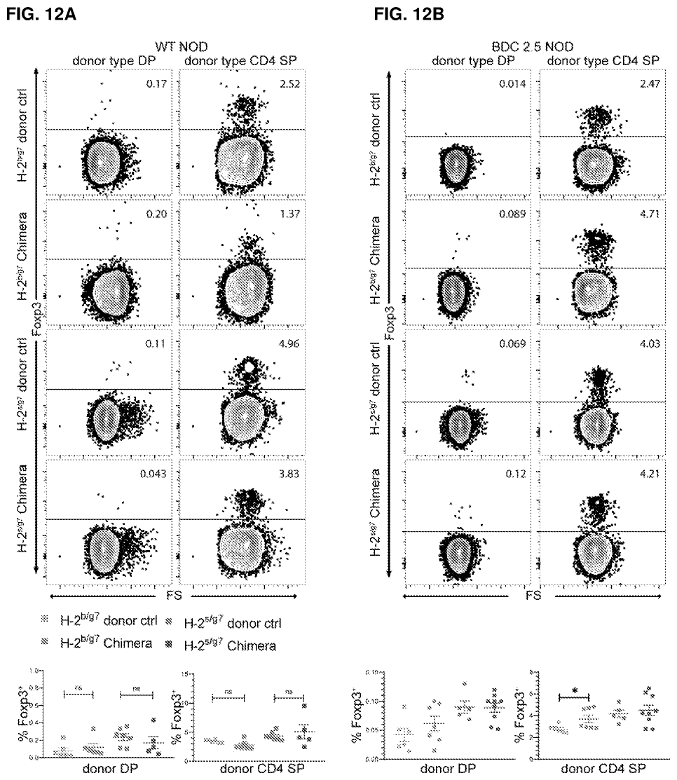

Augmentation of negative selection of conventional thymocytes is often accompanied by enhanced tTreg production (15). As shown in FIG. 11, induction of H-2^{b/g7} or H-2^{s/g7} Haplo-MC increased percentage of Foxp3⁺ tTreg cells among host-type DP and CD4⁺ SP thymocytes in WT NOD mice (FIG. 11A) and increased percentage of Foxp3⁺ tTreg cells among CD4⁺ SP thymocytes in transgenic BDC2.5 NOD mice (FIG. 111B). Foxp3⁺ tTreg cells among DP thymocytes in the mixed chimeric BDC2.5 NOD mice were not measured, due to too few host-type DP thymocytes for reliable analysis as shown in FIG. 9B. Donor-type Treg production was also enhanced in the thymus of transgenic BDC2.5 NOD mice, although not in the thymus of WT NOD mice (FIG. 12). These results indicate that Haplo-MC augments thymic generation of host-type tTreg cells in NOD mice.

### Example 7: Donor-Type DC Subsets are Present in the Thymus of Haplo-MC Mice

There are multiple subsets of CD11 c⁺ DCs in the thymus, including CD11 c⁺B220⁺PDCA-1⁺ plasmacytoid DCs (pDCs), CD8⁺SIRPα⁻ thymus-resident DCs (tDCs), and CD8⁻SIRPα⁺ migratory DCs (mDCs). pDCs and tDCs augment thymic negative selection with limited impact in Treg generation. In contrast, mDCs augment both central negative selection and thymic Treg (tTreg) generation (34-37). As shown in FIG. 11C, all three subsets of donor-type DCs were present in the thymus of the wild-type NOD with Haplo-MC. As compared to control donor, there was a significant increase in CD8⁺ tDCs, but no difference or a reduction in the percentage of pDCs and mDCs (FIG. 11C). Therefore, the increased negative selection and augmented Treg generation in the thymus of Haplo-MC is associated with presence of donor-type DC subsets.

### Example 8: Haplo-MC Augments Reduction of Host-Type CD62L⁻CD44^{hi} Effector Memory T Cells in the Periphery of Both Euthymic and Thymectomized NOD Mice

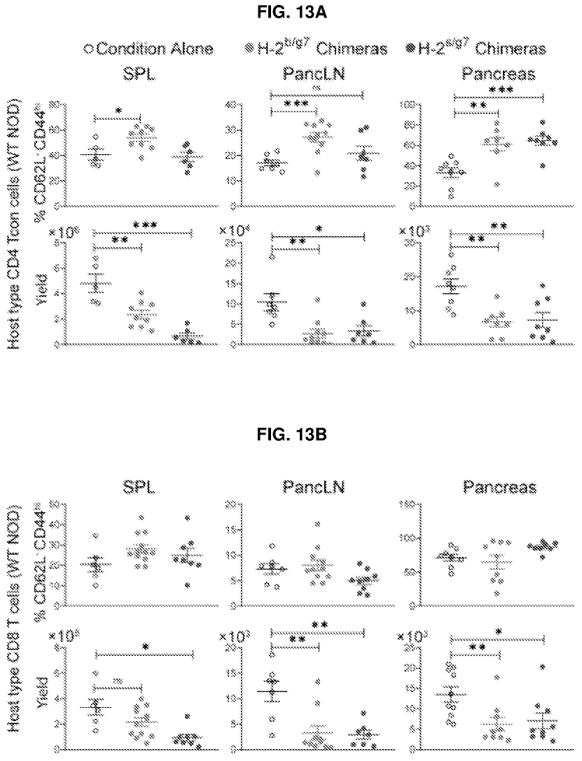

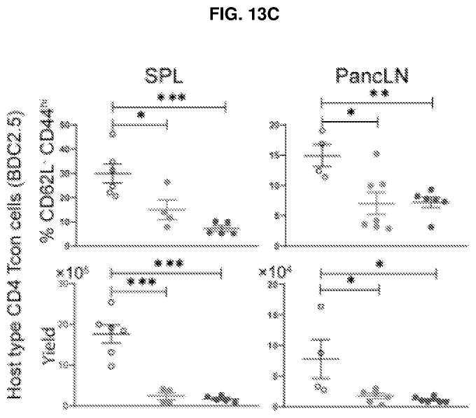

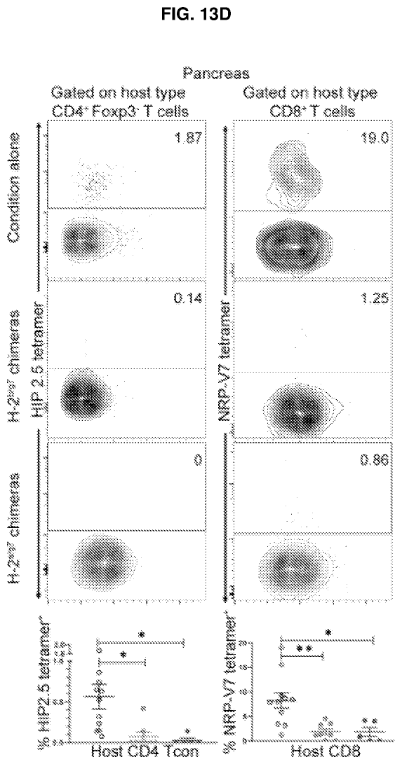

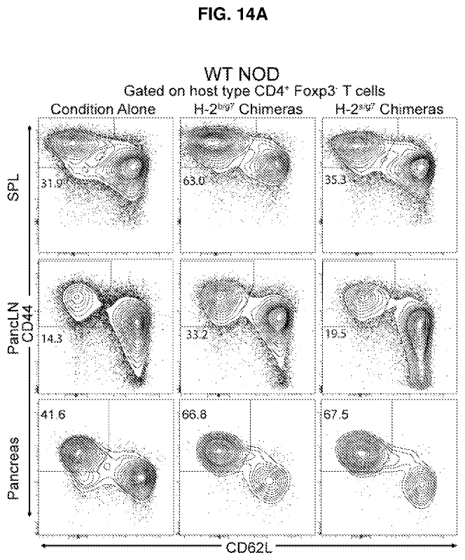

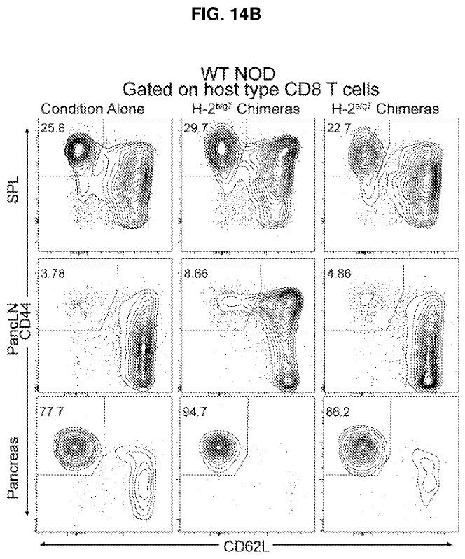

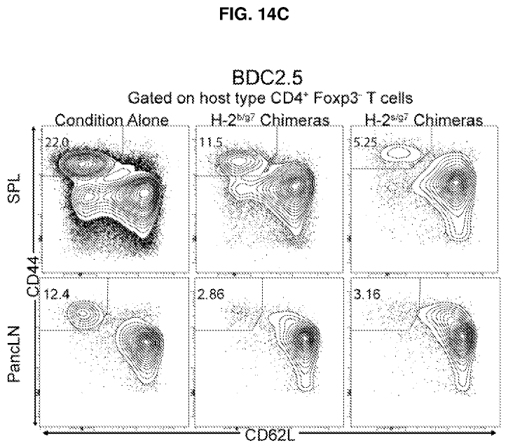

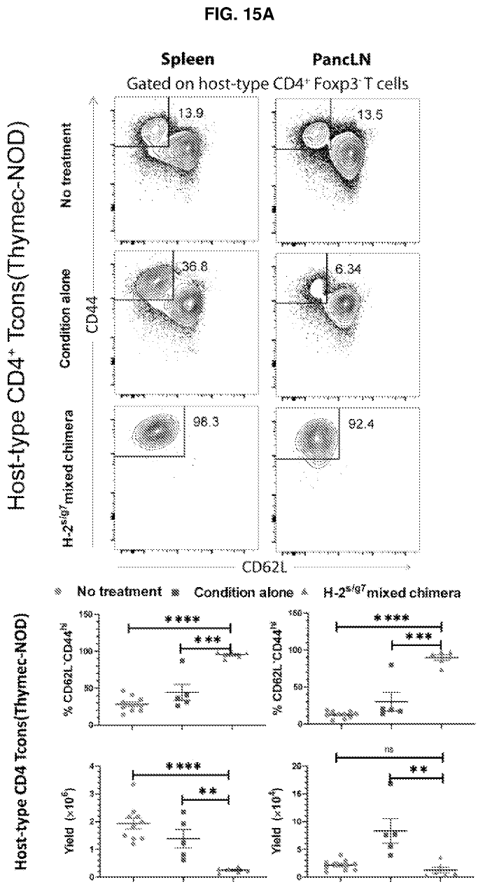

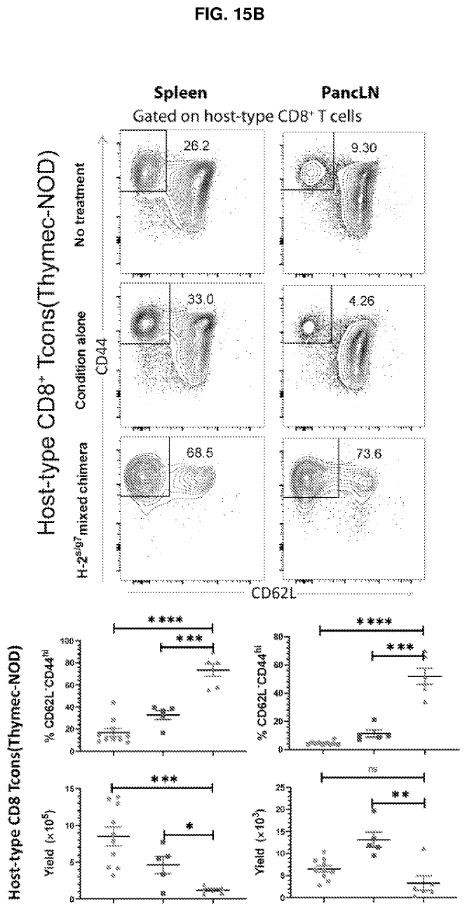

Since H-2^{g/7} and H-2^{s/g7} Haplo-MC eliminated or markedly reduced insulitis in established diabetic NOD mice (FIG. 4), the percentage and yield of host-type CD62L⁻CD44^{hi} effector memory (Tem) cells in the spleen, PancLN and pancreas of Haplo-MC WT NOD mice were compared. Interestingly, Haplo-MC did not reduce but rather increased the percentage of CD62L⁻CD44^{hi} CD4⁺ or CD8⁺ Tem cells in the spleen, PancLN and pancreas of WT NOD mice, however, the yield was markedly reduced (FIGS. 13A-13B and 14A-14B). Similar results were observed in adult-thymectomized NOD mice with Haplo-MC (FIG. 15).

On the other hand, both percentage and yield of the host-type autoreactive CD62L⁻CD44^{hi} CD4⁺ Tem cells in the spleen or PancLN of Haplo-MC transgenic BDC2.5 NOD mice were markedly reduced (FIGS. 13C and 14C). Furthermore, a HIP2.5-tetramer that specifically identifies the chromogranin-proinsulin hybrid peptide-specific autoreactive CD4⁺ T cells (38) and a NRP-V7-tetramer that specifically identifies IGRP₂₀₆₋₂₁₄ peptide-specific autoreactive CD8⁺ T cells (39) were used to measure the changes of the antigen-specific autoreactive Foxp3⁻CD4⁺ and CD8⁺ T cells in the pancreas. Tetramer⁺CD4⁺ or CD8⁺ T cells in WT NOD mice given conditioning alone were only detectable in the pancreas but not in the spleen or PancLN, ˜1% among Foxp3⁻CD4⁺ T and ˜10% among CD8⁺ T cells (FIG. 13D). Both H-2^{g/7} and H-2^{s/g7} Haplo-MC depleted the autoreactive Foxp3⁻CD4⁺ or CD8⁺ T cells in the pancreas of Halo-MC WT NOD mice (FIG. 13D). These results indicate that Haplo-MC preferentially reduces host-type autoreactive Foxp3⁻ conventional T cells in the periphery.

### Example 9: Haplo-MC Augments Expansion of Nrp-1⁺CD73^{hi}FR4^{hi} Anergic CD4⁺ T Cells in the Periphery of Euthymic but not Thymectomized NOD Mice

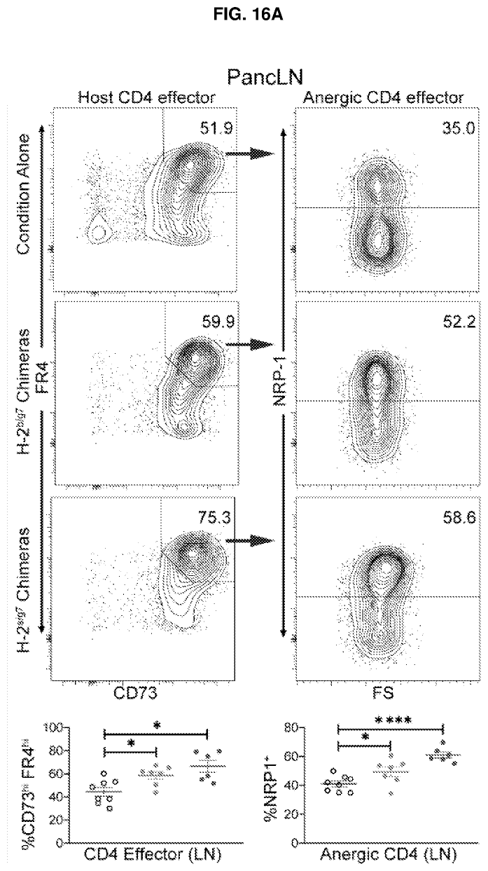

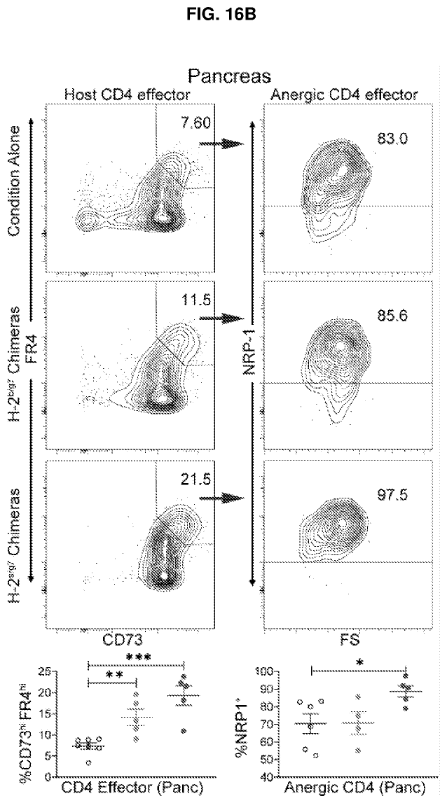

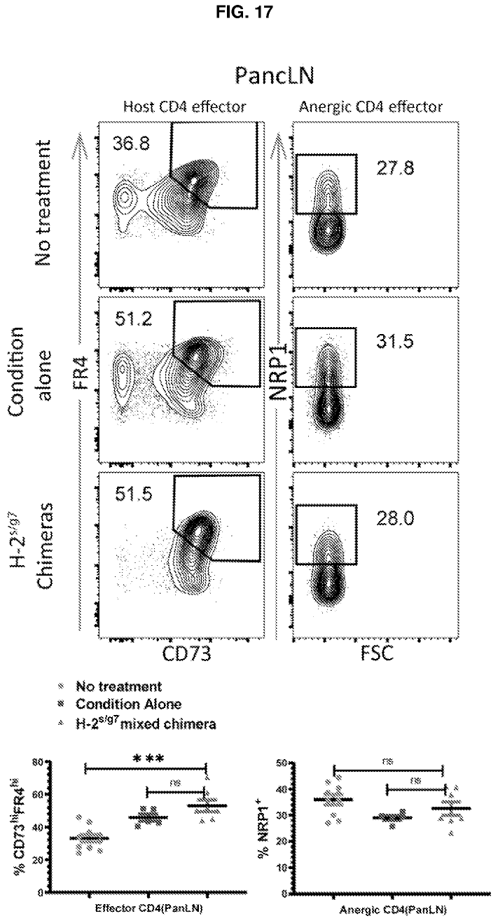

CD73^{hi}FR4^{hi}CD4⁺ T cells in the periphery are anergic T cells (40), and Nrp-1⁺ anergic CD4⁺ T cells can be the precursors of Helios⁻Nrp-1⁺ peripheral Treg (pTreg) cells (41, 42). As compared to control NOD mice, the residual CD4⁺ Tem cells in the PancLN and pancreas of the Haplo-MC NOD mice contained a higher percentage of anergic CD73^{hi}FR4^{hi}CD4⁺ T cells, and higher percentage of Nrp-1⁺ cells among the CD73^{hi}FR4^{hi} Tem cells (FIGS. 16A and 16B). With Thymic-NOD mice, the conditioning alone increased the percentage of CD73^{hi}FR4^{hi} cells among residual host-type CD62L⁻CD44^{hi}CD4⁺ Tem cells in the PancLN as compared to unconditioned mice, and induction of mixed chimerism did not further increase the percentage (FIG. 17). And no difference in the percentage of Nrp-1⁺ cells among the CD73^{hi}FR4^{hi} cells in the mixed chimeras was observed (FIG. 17). These results indicate that residual host-type CD4⁺ T cells in the pancreatic LN and pancreas of both euthymic and thymectomized Haplo-MC NOD mice have enhanced anergy status, but increase of Nrp-1⁺ anergic CD4⁺ T cells is only observed in euthymic Haplo-MC NOD mice.

### Example 10: Haplo-MC Augments Expansion of Host-Type CD62L⁻CD44^{hi} Effector Memory tTreg and Helios⁻Nrp-1⁺ pTreg Cells in the PancLN and Pancreas of Euthymic but not Thymectomized NOD Mice

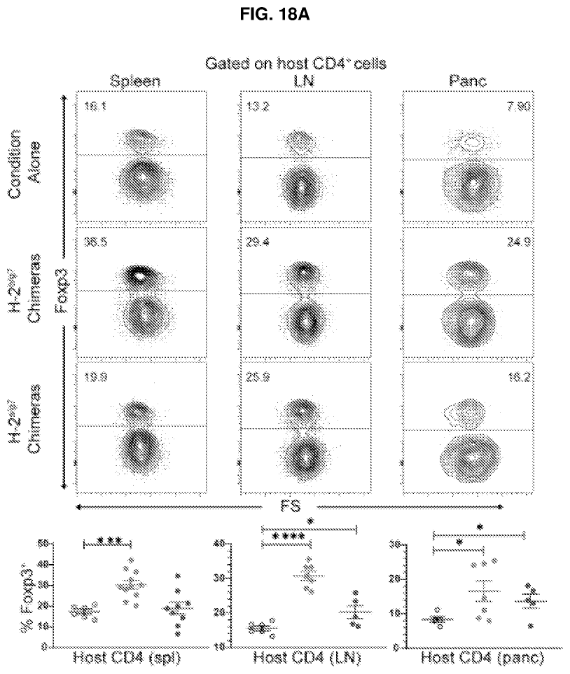

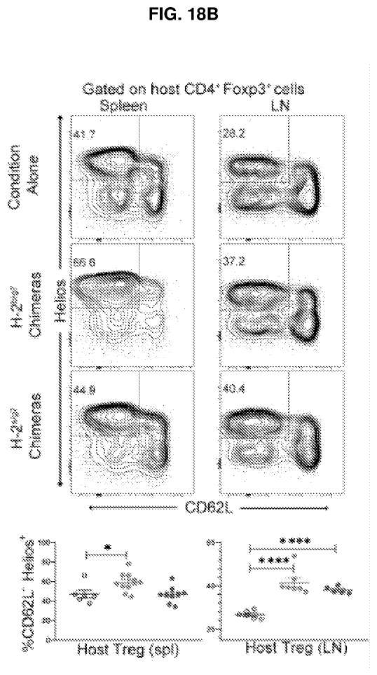

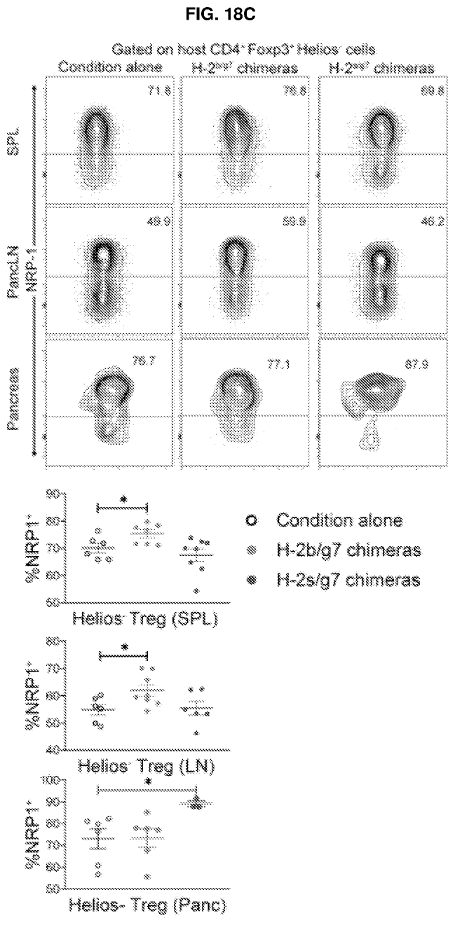

Foxp3⁺ Treg cells in the periphery include thymus-derived Helios⁺ tTreg and peripheral conventional T-derived antigen-specific Helios⁻Nrp-1⁺ pTreg cells (42). tTreg and pTreg cells play important roles in regulating systemic and local autoimmunity, respectively (43). Changes of Treg cells in the spleen reflect systemic, and changes in the organ or organ-draining LN such as PancLN and pancreas reflect local regulation of immune response. Thus, the changes of donor- and host-type Treg subsets were changed in the periphery including spleen, PancLN and pancreas of Haplo-MC NOD mice. The total host-type Treg cells were expanded in the pancreatic LN and pancreas of both H-2^{g/7} and H-2^{s/g7} Haplo-MC, although Treg expansion in the spleen was observed only in H-2^{g/7} but not H-2^{s/g7} mixed chimeras (FIG. 18A). Based on Helios and CD62L staining, significant expansion of CD62L⁻Helios⁺ effector memory tTreg cells in the pancreatic LN of both mixed chimeras as compared to NOD mice given conditioning alone was observed (FIG. 18B).

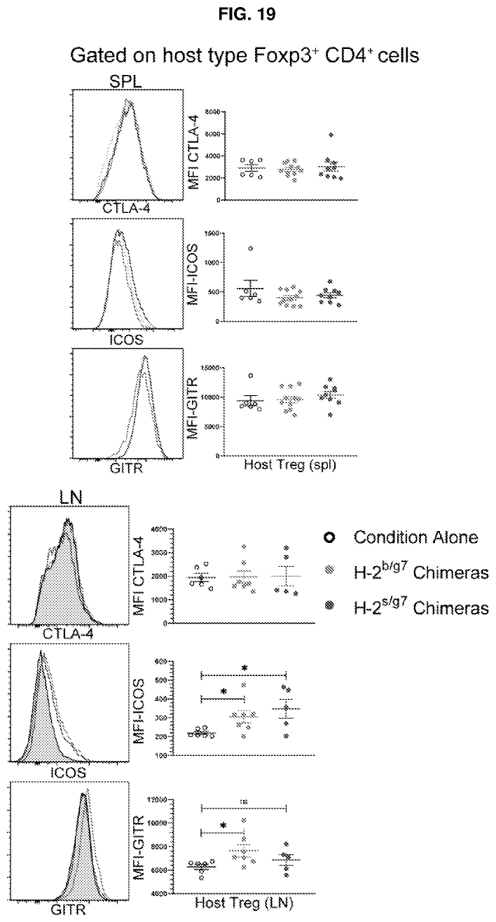

As mentioned above, expansion of Nrp-1⁺CD73^{hi}FR4^{hi}CD4⁺ T cells and the Nrp-1⁺ pTreg precursors, was observed in the Haplo-MC NOD mice (FIG. 16). Thus, the percentages of Nrp-1⁺Helios⁻ pTreg cells in the H-2^{b/g7} and H-2^{s/g7} Haplo-MC were compared. Gating on host-type Helios⁻Foxp3⁺ pTreg cells, there was an increase of Nrp-1⁺ pTreg cells in the spleen and PancLN of H-2^{g/7} mixed chimeras and an increase of Nrp-1⁺ pTreg cells in the pancreas of H-2^{s/g7} mixed chimeras (FIG. 18C). Upregulation of ICOS, GITR and CTLA4 expression is associated with enhanced Treg function (44-47), consistently, host-type Treg cells in the PancLN of mixed chimeras upregulated expression of ICOS and GITR, although no difference in CTLA4 expression was observed (FIG. 19). No difference was observed in Treg expression of ICOS, GITR or CTLA4 in the spleen of the mixed chimeras or control mice (FIG. 19).

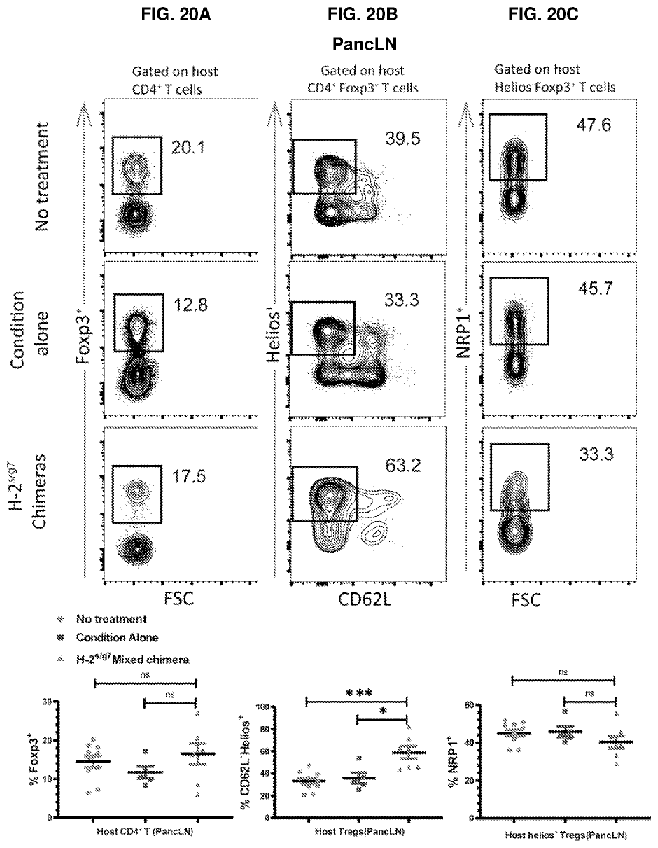

However, compared to Thymec-NOD given conditioning alone, Thymec-NOD mice with Haplo-MC did not show significant difference in the percentage of total Treg cells or host-type Nrp-1⁺Helios⁻ pTreg cells, although they showed an increase in the percentage of Helios⁺CD62L⁻ effector memory tTreg cells among total Treg cells (FIG. 20). Taken collectively, these results indicate that 1) Haplo-MC augments activation and expansion of host-type Helios⁺ tTreg subset in the PancLN and pancreas of NOD mice; and 2) Haplo-MC also augments expansion of Helios⁻Nrp-1⁺ pTreg cells in euthymic but not thymectomized Haplo-MC NOD mice.

### Example 11: Haplo-MC Augments Expansion of Donor-Type CD62L⁻CD44^{hi} Effector Memory tTreg in the PancLN and Pancreas of Euthymic and Thymectomized NOD Mice

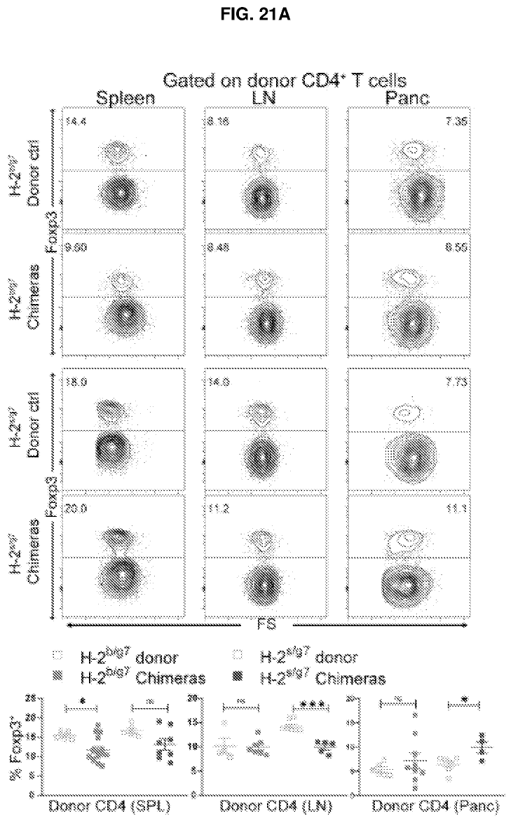

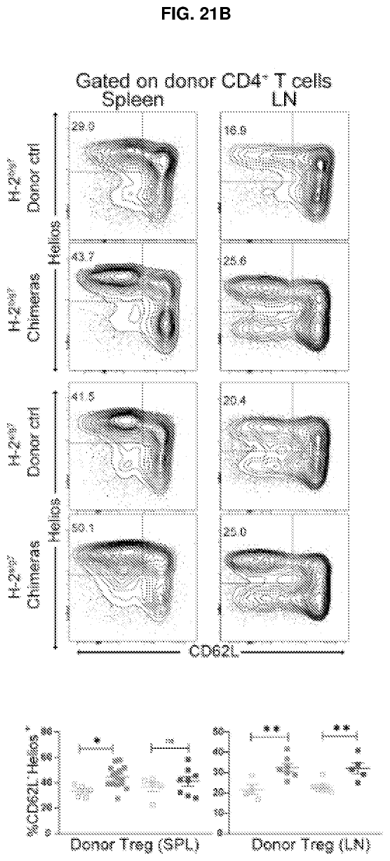

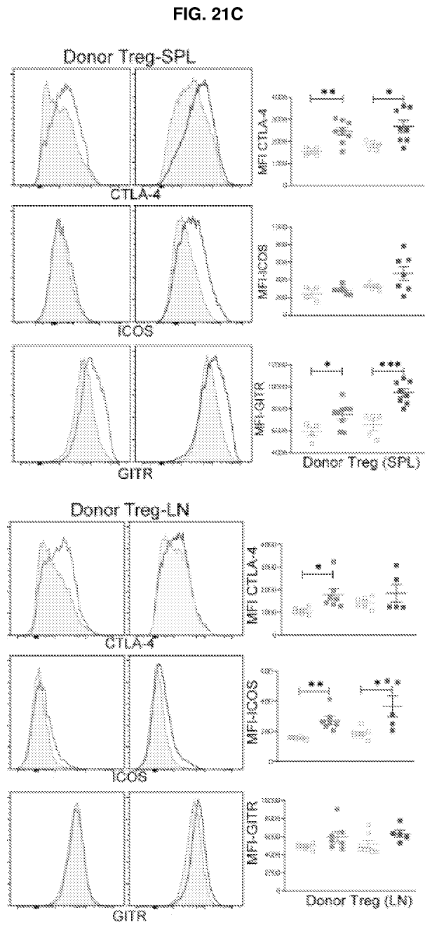

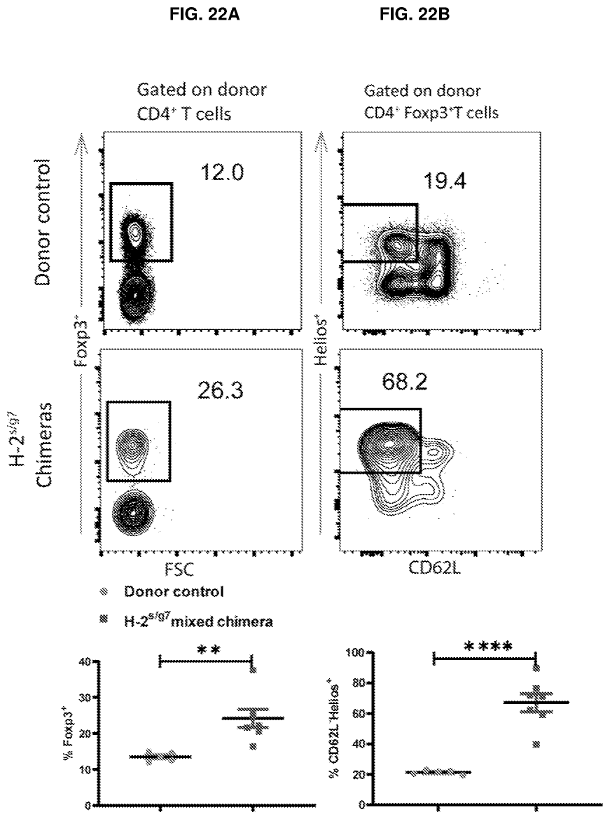

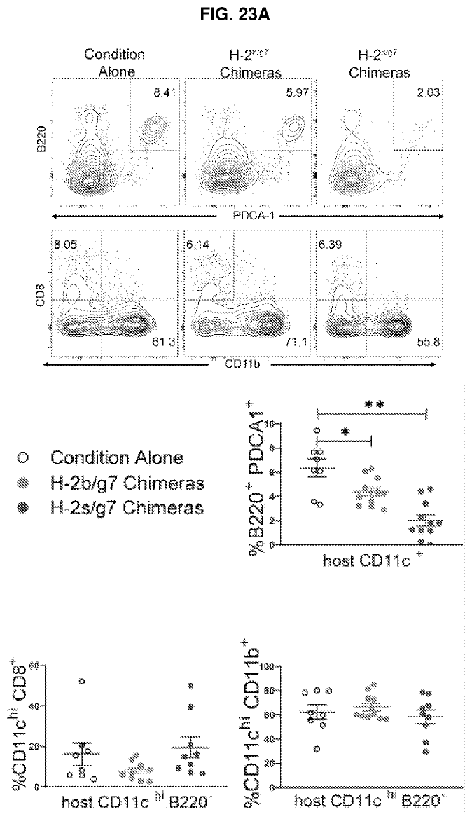

Donor-type Treg cells were present in the spleen, PancLN and pancreas of both H-2^{g/7} and H-2^{s/g7} Haplo-MC. As compared to control donor mice, the percentage of total Treg of Haplo-MC was similar in the spleen and variable in the PancLN and pancreas (FIG. 21A). However, the percentage of CD62L⁻Helios⁺ effector memory tTreg cells in the Haplo-MC was increased in both spleen and PancLN (FIG. 21B). Furthermore, donor-type Treg cells in the spleen and/or PancLN of Haplo-MC upregulated expression of CTLA4, although expression of ICOS or GITR was variable (FIG. 21C). Similarly, as compared to donor control, there was a marked increase of donor-type total Treg and Helios⁺CD62L⁻ effector memory tTreg cells in the PancLN of Haplo-MC Thymec-NOD mice (FIG. 22). These results indicate that Haplo-MC augments activation and expansion of donor-type tTreg cells in the periphery of both euthymic and thymectomized Haplo-MC NOD mice.

### Example 12: Haplo-MC Upregulates Host-Type pDC Expression of PD-L1 in Euthymic but not Thymectomized NOD Mice

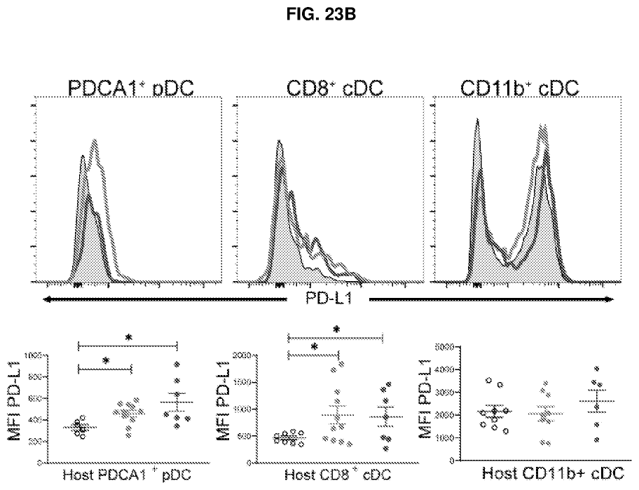

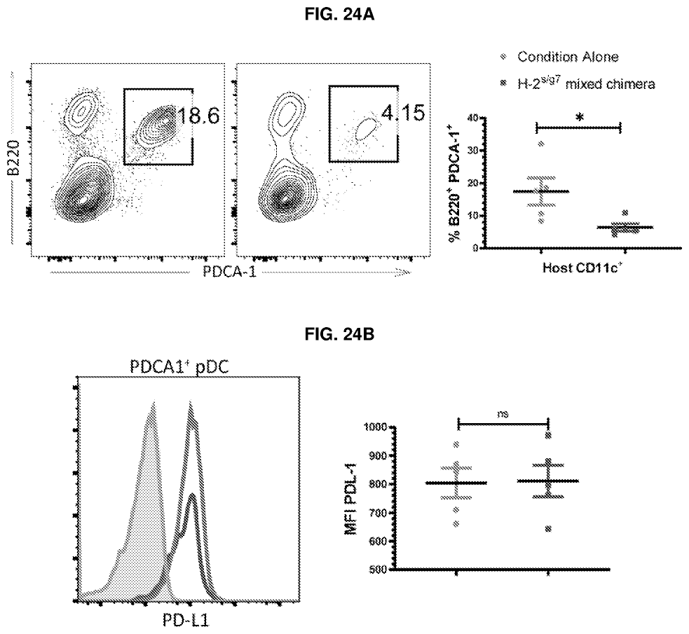

Peripheral tolerance is associated with tolerogenic DCs, especially pDCs that express high levels of PD-L1 (48, 49), and loss of tolerogenic features of pDC in the periphery plays an important role in T1 D pathogenesis (50, 51). Thus, changes of host-type DCs as well as their expression of PD-L1 in the spleen of mixed chimeras were measured. Among host-type DCs in both H-2^{g/7} and H-2^{s/g7} Haplo-MC, there was a marked reduction in percentage of CD11 c⁺B220⁺PDCA-1⁺ pDC among total host-type DCs, especially in the H-2^{s/g7} mixed chimeras, as compared to that of control mice given conditioning alone, although no significant changes in the percentage of CD8⁺ or CD11b⁺ DC subsets (FIG. 23A). In contrast, the residual pDCs in both mixed chimeras upregulated expression of PD-L1, as did CD8⁺ DC subset, but not CD11b⁺ DC subset (FIG. 23B). Interestingly, although there was a marked reduction of pDC in the spleen of Haplo-MC of Thymec-NOD, the residual pDC did not upregulate their expression of PD-L1 as compared to conditioning alone (FIG. 24). These results indicate that induction of Haplo-MC reduces host-type pDCs in both euthymic and thymectomized NOD mice, but Haplo-MC augments the residual pDCs upregulate their expression of PD-L1 in the euthymic but not thymectomized mice.

### Example 13: Maintenance of Peripheral Tolerance of Residual Host-Type Autoreactive T Cells in the Euthymic Haplo-MC Mice Requires Both Donor- and Host-Type Foxp3⁺ Treg Cells

Since there was an expansion of donor- and host-type Treg effector memory cells in both H-2^{g/7} and H-2^{s/g7} mixed chimeric NOD (FIGS. 18 and 21), whether those Treg cells were required for maintaining peripheral tolerance was tested by using Foxp3^{DTR} expression in either donor- or host-type Treg cells in H-2^{g/7} mixed chimeric NOD mice, as depicted in FIG. 25A. Depletion of Treg cells was induced by injection of DT every 3 days for 21 days, starting at 45-60 days after induction of mixed chimerism, as described in the materials and methods. Injection of DT specifically reduced donor-type Treg by ˜95% and reduced host-type Treg by ˜90% (FIGS. 25B and 26). Depletion of donor-type or host-type Treg cells induced significant but moderate recurrence of insulitis, without causing hyperglycemia (FIG. 25C). Simultaneous depletion of both donor- and host-type Treg cells did not appear to significantly enhance the insulitis, but because the treatment led to rapid decline of health, and the mice died or became very sick without hyperglycemia before completion of treatment, the results could not be used for comparison. Therefore, comparison with depletion of donor-type versus depletion of host-type Treg cells was made. Depletion of donor-type Treg cells but not depletion of host-type Treg cells led to increase in percentage of host-type CD4⁺ and CD8⁺CD62L⁻CD44⁺ Tcon effector memory cells in the PancLN (FIG. 25D). In contrast, depletion of host-type but not donor-type Treg cells led to decrease in percentage of CD73^{hi}FR4^{hi} anergic CD4⁺ Tcon and IL-7Rα⁻PD-1^{hi} anergic/exhausted CD8⁺ Tcon cells (FIG. 25E). These results indicate that both donor- and host-type Treg cells contribute to maintenance of peripheral tolerance of residual autoreactive T cells, although each have a different functional effect.

### Example 14: Maintenance of Peripheral Tolerance of Residual Host-Type Autoreactive T Cells Requires Host-Hematopoietic Cell Expression of PD-L1

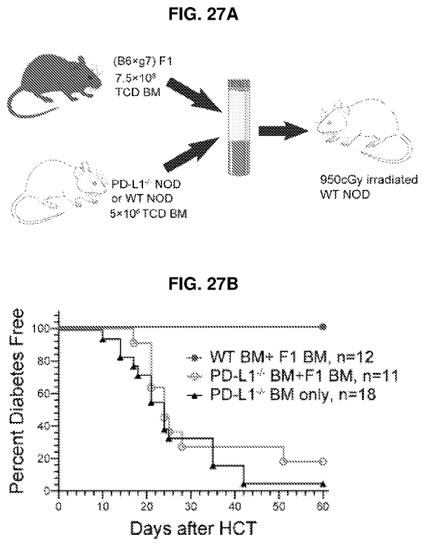

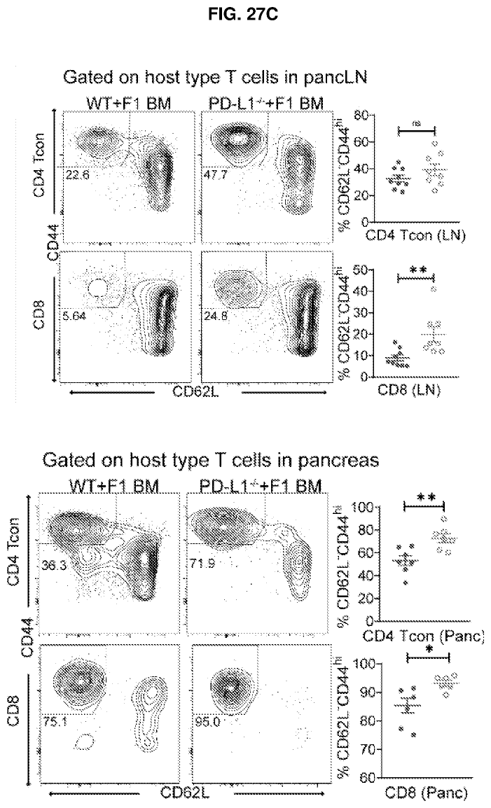

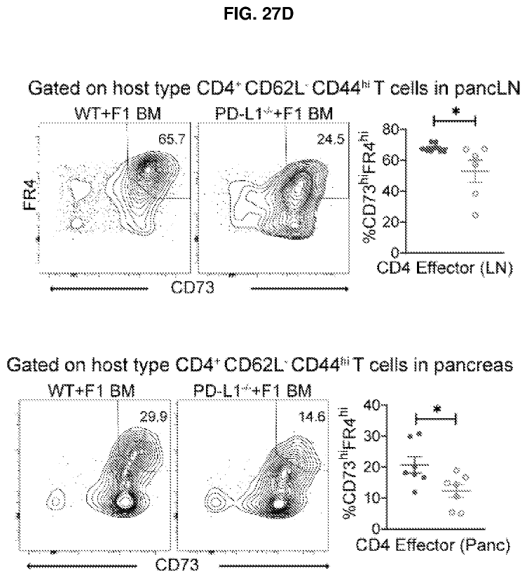

Because host-type DCs, especially pDCs, in the H-2^{g/7} and H-2^{s/g7} Haplo-MC euthymic NOD mice expressed higher levels of PD-L1 as compared to mice given conditioning alone (FIG. 23), whether host DC expression of PD-L1 was required for maintaining the peripheral tolerance was tested using H-2^{b/g7} mixed chimeric NOD mice. Parenchymal cell expression of PD-L1 was reported to play a critical role in prevention of T1 D in NOD mice (52). The role of host-type DC expression of PD-L1 on maintaining peripheral tolerance in the presence of host-parenchymal tissue expression of PD-L1 was evaluated. Accordingly, Haplo-MC was established by co-injection of donor-type TCD-BM from H-2^{g/7} F1 donor mice and host-type TCD-BM from WT or PD-1^{-/-} NOD mice into lethally irradiated WT NOD mice, as depicted in FIG. 27A. The control NOD recipients were given PD-L1^{-/-}-NOD TCD-BM alone.

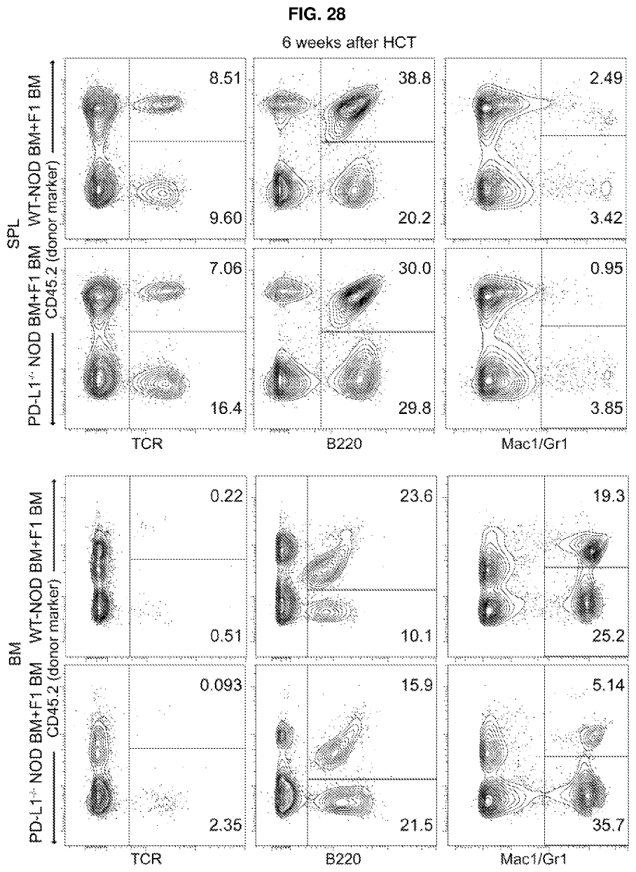

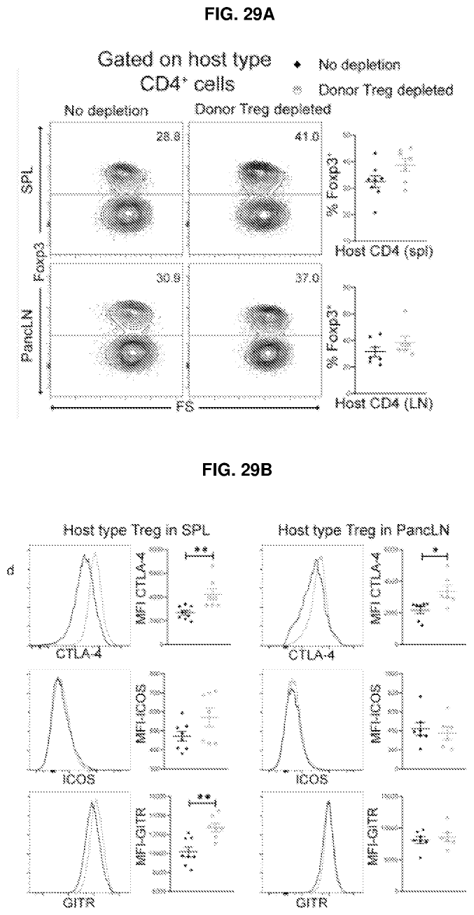

The NOD recipients with TCD-BM from H-2^{g/7} F1 donor and TCD-BM from syngeneic WT or PD-L1^{-/-} NOD mice developed stable mixed chimerism (FIG. 28). While none (0/12) of the H-2^{g/7} mixed chimeras that received PD-L1^{+/+}NOD TCD-BM (PD-L1^{+/+} chimeras) developed T1 D or hyperglycemia, 82% ( 9/11) of the H-2^{b/g7} mixed chimeras that received PD-L1^{-/-} NOD TCD-BM (PD-L1^{-/-} chimeras) developed T1 D with hyperglycemia, and 94% ( 17/18) NOD recipients given PD-L1^{-/-} NOD TCD-BM alone (PD-L1^{-/-} NOD) developed T1 D with hyperglycemia (FIG. 27B). Furthermore, as compared with PD-L1^{+/+} mixed chimeras without T1 D, PD-L1^{-/-} mixed chimeras with T1 D showed expansion of host-type CD4⁺ and CD8⁺ T effector cells in the pancreatic LN and pancreas (FIG. 27C). Those T effector cells had a decrease in percentage of anergic CD73^{hi}FR4^{hi}CD4⁺ T cells (FIG. 27D). These results indicate that host-type hematopoietic cell expression of PD-L1 is required for maintaining peripheral tolerance of residual autoreactive T cells in Haplo-MC euthymic NOD mice.

### Example 15: There is a Mutual Influence and Compensatory Role Between Donor- and Host-Type Treg Cells in Euthymic Haplo-MC NOD Mice

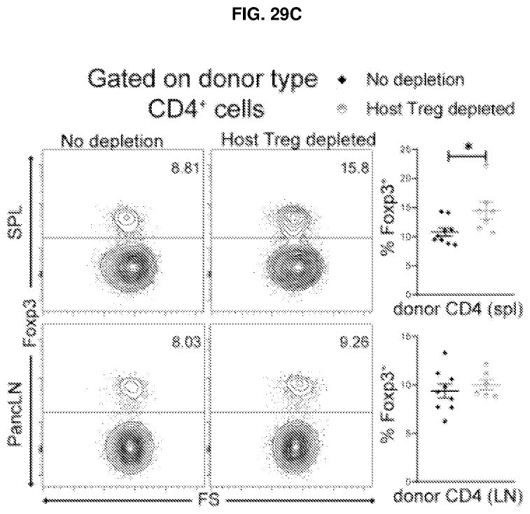

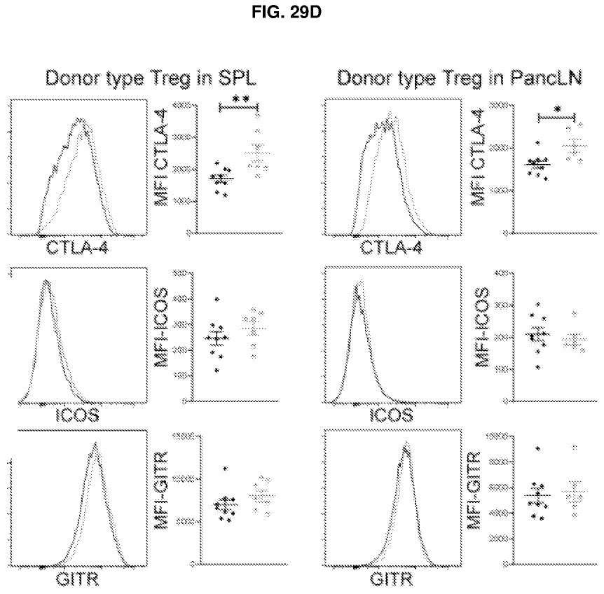

Both donor- and host-type Treg cells were activated in the Haplo-MC NOD mice, as indicated by the relative increase of CD62L⁻ effector memory Treg cells, although they showed different changes in surface receptors: donor-type Treg cells upregulated expression of CTLA4, but host-type Treg cells upregulated expression of ICOS and GITR (FIGS. 18, 19, and 21). Next, whether there is a mutual influence between donor- and host-type Treg cells in the Haplo-MC NOD mice was evaluated. Depletion of donor-type Treg cells led to slight increase in the percentage of host-type Treg cells and significant upregulation of expression of CTLA4 in the spleen and PancLN (FIGS. 29A & 29B). However, upregulation of expression of ICOS and GITR was observed only in the spleen but not in the PancLN (FIG. 29B). In contrast, depletion of host-type Treg cells led to significant expansion of donor-type Treg cells and their upregulation of expression of CTLA4 in the spleen but not in the PancLN. In addition, no significant changes in ICOS and GITR expression in the spleen or PancLN were observed (FIGS. 29C & 29D). These results suggest that the regulatory emphasis of donor- and host-type Treg cells differs: donor-type Treg cells are more involved in regulating systemic immune response such as in the spleen, and host-type Treg cells are more involved in regulating local immune response such as in the PancLN. These observations may also provide an explanation to why depletion of donor- or host-type Treg cell alone did not cause overt insulitis or hyperglycemia in the Haplo-MC NOD mice.

### Example 16: Donor- and Host-Type tTreg Cells are Required for Upregulating Host-Type pDC Expression of PD-L1 that Augments Expansion of Host-Type and Donor-Type Nrp-1⁺Helios⁻ pTreg Cells

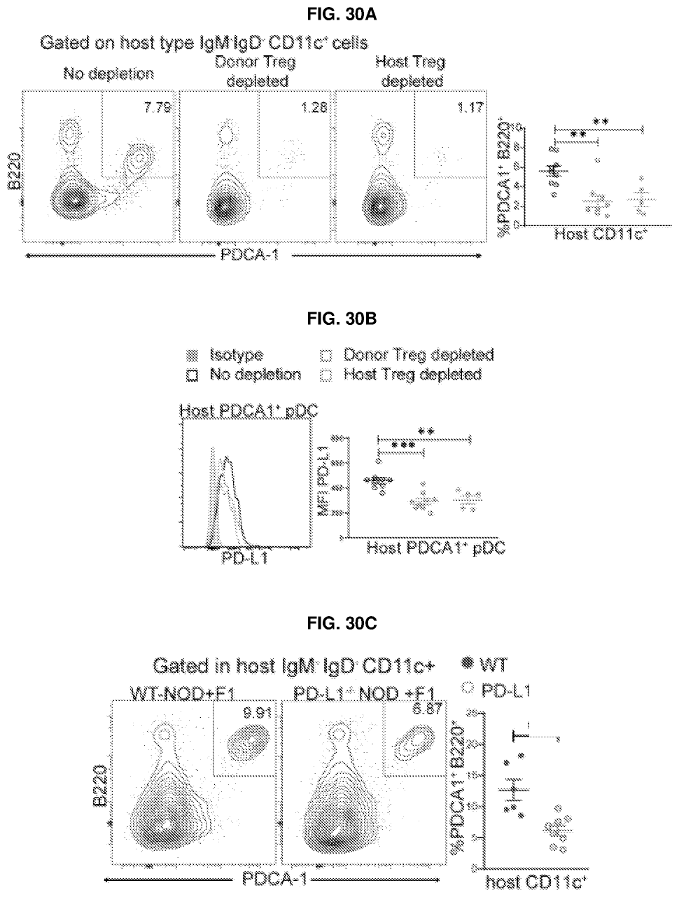

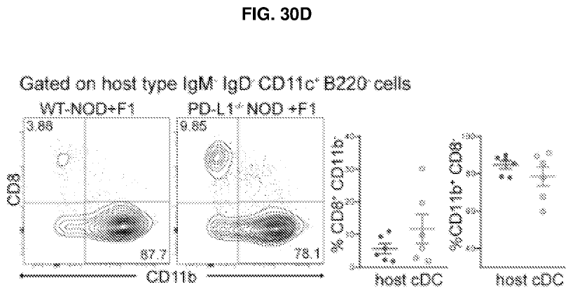

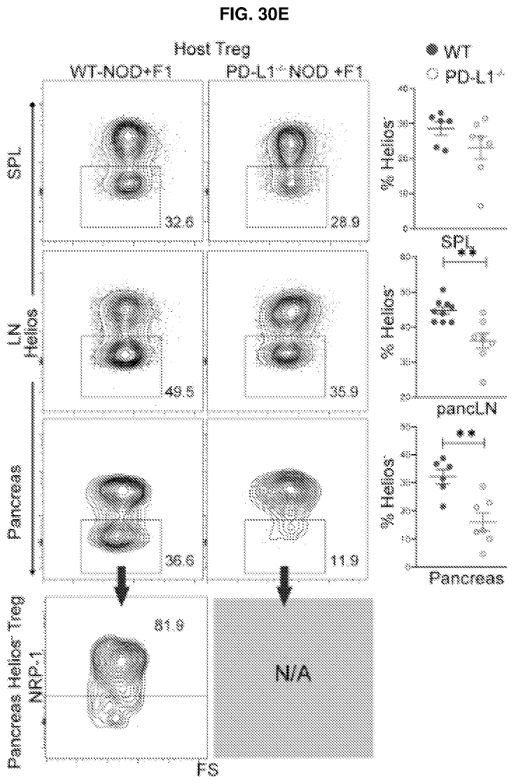

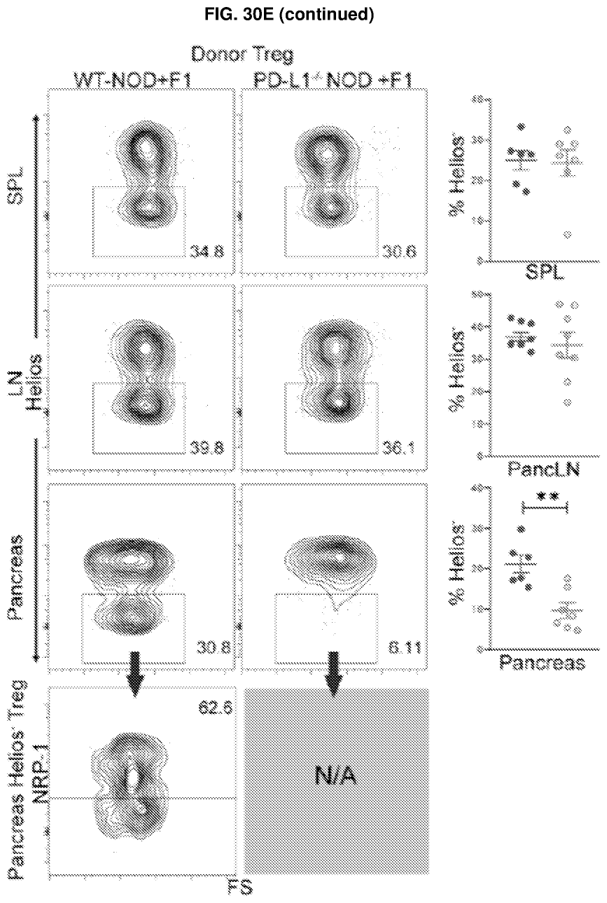

Because host-type pDCs were found to upregulate expression of PD-L1 in Haplo-MC euthymic NOD mice (FIG. 23), the impact of depletion of Treg cells on the host-type pDC expression of PD-L1 was analyzed. Interestingly, depletion of either donor-type or host-type Treg cells led to a decrease in the percentage of host-type B220⁺PDCA-1⁺ pDCs (FIG. 30A) as well as their down-regulation of expression of PD-L1 (FIG. 30B). These results suggest that donor- and host-type Treg cells can augment host-type pDC expansion and their expression of PD-L1.

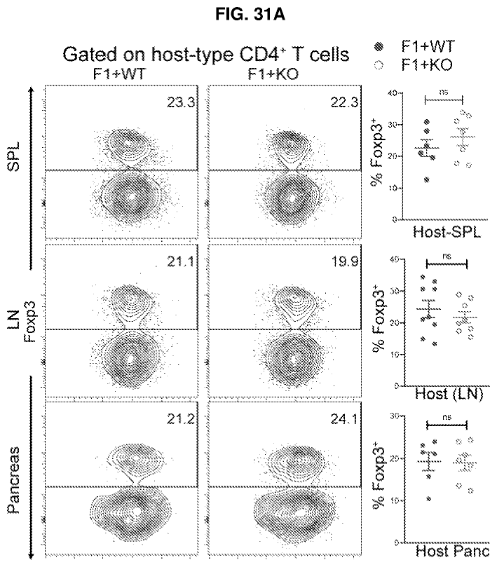

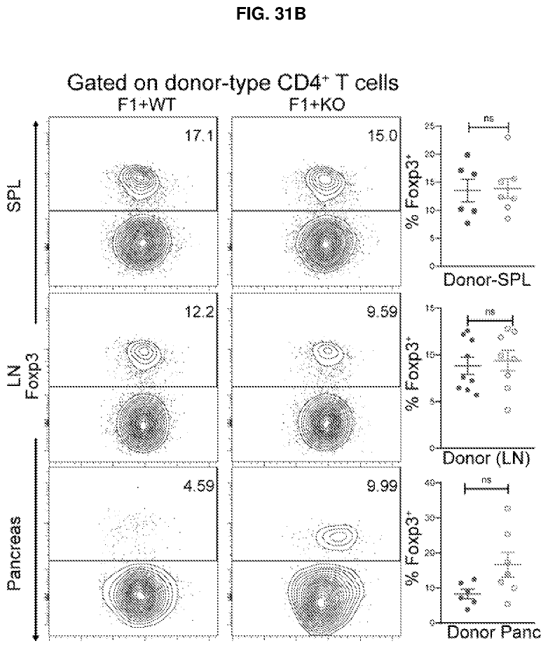

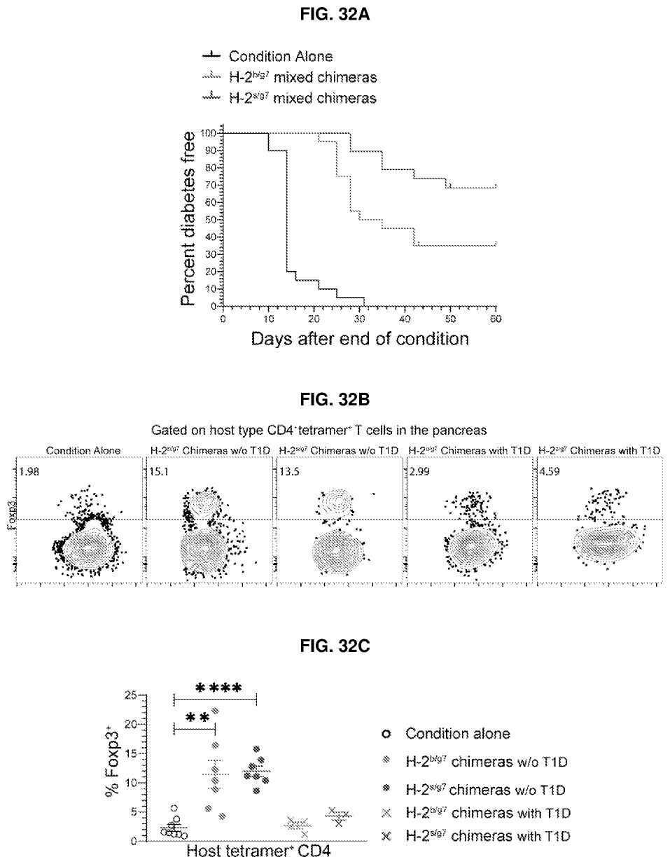

Furthermore, the impact of PD-L1 expression by host-type hematopoietic cells on expansion of host-type pDC and Treg cells was evaluated. PD-L1 deficiency in host-type hematopoietic cells led to a marked decrease in the percentage of host-type pDCs (FIG. 30C), although no reduction in CD8⁺ lymphoid or CD11b⁺ myeloid DC subsets was observed (FIG. 30D). Also, PD-L1 deficiency in host-type hematopoietic cells resulted in no changes in the total percentage of host- and donor-type Foxp3⁺ Treg cells in the spleen, PancLN or pancreas (FIGS. 31A & 31B). However, the PD-L1 deficiency in host-type hematopoietic cells resulted in a marked reduction in the percentage of host-type Helios⁻ pTreg cells that are predominantly Nrp-1⁺ in the PancLN and pancreas as well as marked reduction of donor-type Helios⁻ pTreg cells in the pancreas (FIG. 30E). Additionally, expansion of antigen-specific Treg cells in the pancreas of Haplo-MC BDC2.5 NOD mice was associated with effective prevention of T1 D (FIG. 32). These results indicate that 1) host-type pDC expression of PD-L1 play a critical role in expansion of host-type Helios⁻Nrp-1⁺ pTreg cells in the PancLN and pancreas of Haplo-MC euthymic NOD mice; and 2) autoantigen-specific pTreg cells may play an important role in controlling residual autoreactive T cells in the Haplo-MC euthymic NOD mice.

### REFERENCES

The references, patents and published patent applications listed below, and all references cited in the specification above are hereby incorporated by reference in their entireties, as if fully set forth herein.
1. Kanakry C G, Fuchs E J, and Luznik L. Modern approaches to HLA-haploidentical blood or marrow transplantation. Nature reviews Clinical oncology. 2016; 13(1):10-24.
2. Strober S. Use of hematopoietic cell transplants to achieve tolerance in patients with solid organ transplants. Blood. 2016; 127(12):1539-43.
3. Chen Y B, Elias N, Heher E, McCune J S, Collier K, Li S, Del Rio C, El-Jawahri A, Williams W, Tolkoff-Rubin N, et al. Haploidentical hematopoietic cell and kidney transplantation for hematological malignancies and end-stage renal failure. Blood. 2019; 134(2):211-5.
4. Kawai T, Sachs D H, Sykes M, Cosimi A B, and Immune Tolerance N. HLA-mismatched renal transplantation without maintenance immunosuppression. The New England journal of medicine. 2013; 368(19):1850-2.
5. Leventhal J R, Elliott M J, Yolcu E S, Bozulic L D, Tollerud D J, Mathew J M, Konieczna I, Ison M G, Galvin J, Mehta J, et al. Immune reconstitution/immunocompetence in recipients of kidney plus hematopoietic stem/facilitating cell transplants. Transplantation. 2015; 99(2):288-98.
6. Racine J, Wang M, Zhang C, Lin C L, Liu H, Todorov I, Atkinson M, and Zeng D. Induction of mixed chimerism with MHC-mismatched but not matched bone marrow transplants results in thymic deletion of host-type autoreactive T-cells in NOD mice. Diabetes. 2011; 60(2):555-64.
7. Scandling J D, Busque S, Shizuru J A, Lowsky R, Hoppe R, Dejbakhsh-Jones S, Jensen K, Shori A, Strober J A, Lavori P, et al. Chimerism, graft survival, and withdrawal of immunosuppressive drugs in HLA matched and mismatched patients after living donor kidney and hematopoietic cell transplantation. American Journal of Transplantation. 2015; 15(3):695-704.
8. Vanikar A V, Modi P R, Patel R D, Kanodia K V, Shah V R, Trivedi V B, and Trivedi HL. Hematopoietic stem cell transplantation in autoimmune diseases: the Ahmedabad experience. Transplantation proceedings. 2007; 39(3):703-8.
9. Niu H, Fang G, Tang Y, Xie L, Yang H, Morel L, Diamond B, and Zou Y R. The function of hematopoietic stem cells is altered by both genetic and inflammatory factors in lupus mice. Blood. 2013; 121(11):1986-94.
10. Nikolic T, Bunk M, Drexhage H A, and Leenen P J. Bone marrow precursors of nonobese diabetic mice develop into defective macrophage-like dendritic cells in vitro. Journal of immunology. 2004; 173(7):4342-51.
11. Lampeter E F, Homberg M, Quabeck K, Schaefer U W, Wernet P, Bertrams J, Grosse-Wilde H, Gries F A, and Kolb H. Transfer of insulin-dependent diabetes between HLA-identical siblings by bone marrow transplantation. Lancet. 1993; 341(8855):1243-4.
12. Sykes M, and Nikolic B. Treatment of severe autoimmune disease by stem-cell transplantation. Nature. 2005; 435(7042):620-7.
13. Zeng D. Bridge between type 1 diabetes in mouse and man. Proceedings of the National Academy of Sciences of the United States of America. 2017; 114(41):10821-3.
14. Pearson J A, Wong F S, and Wen L. The importance of the Non Obese Diabetic (NOD) mouse model in autoimmune diabetes. Journal of autoimmunity. 2016; 66(76-88.
15. Klein L, Kyewski B, Allen P M, and Hogquist K A. Positive and negative selection of the T cell repertoire: what thymocytes see (and don't see). Nature reviews Immunology. 2014; 14(6):377-91.
16. Unanue E R. Antigen presentation in the autoimmune diabetes of the NOD mouse. Annual review of immunology. 2014; 32(579-608.
17. Markees T G, Serreze D V, Phillips N E, Sorli C H, Gordon E J, Shultz L D, Noelle R J, Woda B A, Greiner D L, Mordes J P, et al. NOD mice have a generalized defect in their response to transplantation tolerance induction. Diabetes. 1999; 48(5):967-74.
18. Liang Y, Huang T, Zhang C, Todorov I, Atkinson M, Kandeel F, Forman S, and Zeng D. Donor C D8+ T cells facilitate induction of chimerism and tolerance without GVHD in autoimmune NOD mice conditioned with anti-CD3 mAb. Blood. 2005; 105(5):2180-8.
19. Wang M, Racine J J, Song X, Li X, Nair I, Liu H, Avakian-Mansoorian A, Johnston H F, Liu C, Shen C, et al. Mixed Chimerism and Growth Factors Augment Cell Regeneration and Reverse Late-Stage Type 1 Diabetes. Science translational medicine. 2012; 4(133):133ra59-ra59.
20. Zhang C, Todorov I, Lin C-L, Atkinson M, Kandeel F, Forman S, and Zeng D. Elimination of insulitis and augmentation of islet beta cell regeneration via induction of chimerism in overtly diabetic NOD mice. Proceedings of the National Academy of Sciences of the United States of America. 2007; 104(7):2337-42.
21. Li N, Zhao D, Kirschbaum M, Zhang C, Lin C L, Todorov I, Kandeel F, Forman S, and Zeng D. HDAC inhibitor reduces cytokine storm and facilitates induction of chimerism that reverses lupus in anti-CD3 conditioning regimen. Proceedings of the National Academy of Sciences of the United States of America. 2008; 105(12):4796-801.
22. Wu L, Li N, Zhang M, Xue S-L, Cassady K, Lin Q, Riggs A D, and Zeng D. MHC-mismatched mixed chimerism augments thymic regulatory T-cell production and prevents relapse of EAE in mice. Proceedings of the National Academy of Sciences. 2015; 112(52):15994-9.
23. Schmidt D, Verdaguer J, Averill N, and Santamaria P. A mechanism for the major histocompatibility complex-linked resistance to autoimmunity. The Journal of experimental medicine. 1997; 186(7):1059-75.
24. Mariotti J, Taylor J, Massey P R, Ryan K, Foley J, Buxhoeveden N, Felizardo T C, Amarnath S, Mossoba M E, and Fowler D H. The pentostatin plus cyclophosphamide nonmyeloablative regimen induces durable host T cell functional deficits and prevents murine marrow allograft rejection. Biology of blood and marrow transplantation: journal of the American Society for Blood and Marrow Transplantation. 2011; 17(5):620-31.
25. Ni X, Song Q, Cassady K, Deng R, Jin H, Zhang M, Dong H, Forman S, Martin P J, Chen Y-Z Z, et al. PD-L1 interacts with CD80 to regulate graft-versus-leukemia activity of donor CD8+ T cells. Journal of Clinical Investigation. 2017; 127(5):1960-77.
26. Haller M J, Schatz D A, Skyler J S, Krischer J P, Bundy B N, Miller J L, Atkinson M A, Becker D J, Baidal D, DiMeglio L A, et al. Low-Dose Anti-Thymocyte Globulin (ATG) Preserves beta-Cell Function and Improves HbA1c in New-Onset Type 1 Diabetes. Diabetes care. 2018; 41(9):1917-25.
27. Haller M J, Gitelman S E, Gottlieb P A, Michels A W, Rosenthal S M, Shuster J J, Zou B, Brusko T_{M}, Hulme M A, Wasserfall C H, et al. Anti-thymocyte globulin/G-CSF treatment preserves beta cell function in patients with established type 1 diabetes. The Journal of clinical investigation. 2015; 125(1):448-55.
28. Dardenne M, Lepault F, Bendelac A, and Bach J F. Acceleration of the onset of diabetes in NOD mice by thymectomy at weaning. Eur J Immunol. 1989; 19(5):889-95.
29. Sykes M, Sheard M A, and Sachs D H. Effects of T cell depletion in radiation bone marrow chimeras. II. Requirement for allogeneic T cells in the reconstituting bone marrow inoculum for subsequent resistance to breaking of tolerance. The Journal of experimental medicine. 1988; 168(2):661-73.
30. Zucchelli S, Holler P, Yamagata T, Roy M, Benoist C, and Mathis D. Defective central tolerance induction in NOD mice: genomics and genetics. Immunity. 2005; 22(3):385-96.
31. Lesage S, Hartley S B, Akkaraju S, Wilson J, Townsend M, and Goodnow C C. Failure to Censor Forbidden Clones of CD4 T Cells in Autoimmune Diabetes. Journal of Experimental Medicine. 2002; 196(9):1175-88.
32. Racine J J, Zhang M, Wang M, Morales W, Shen C, and Zeng D. MHC-mismatched mixed chimerism mediates thymic deletion of cross-reactive autoreactive T cells and prevents insulitis in nonobese diabetic mice. J Immunol. 2015; 194(1):407-17.
33. Kim S M, Bhonsle L, Besgen P, Nickel J, Backes A, Held K, Vollmer S, Dornmair K, and Prinz J C. Analysis of the paired TCR alpha- and beta-chains of single human T cells. PLoS One. 2012; 7(5):e37338.
34. Herbin O, Bonito A J, Jeong S, Weinstein E G, Rahman A H, Xiong H, Merad M, and Alexandropoulos K. Medullary thymic epithelial cells and CD8alpha(+) dendritic cells coordinately regulate central tolerance but CD8alpha(+) cells are dispensable for thymic regulatory T cell production. Journal of autoimmunity. 2016; 75(141-9.
35. Hadeiba H, Lahl K, Edalati A, Oderup C, Habtezion A, Pachynski R, Nguyen L, Ghodsi A, Adler S, and Butcher E C. Plasmacytoid Dendritic Cells Transport Peripheral Antigens to the Thymus to Promote Central Tolerance. Immunity. 2012; 36(3):438-50.
36. Baba T, Nakamoto Y, and Mukaida N. Crucial contribution of thymic Sirp alpha+ conventional dendritic cells to central tolerance against blood-borne antigens in a CCR2-dependent manner. Journal of immunology. 2009; 183(5):3053-63.
37. Leventhal D S, Gilmore D C, Berger J M, Nishi S, Lee V, Malchow S, Kline D E, Kline J, Vander Griend D J, Huang H, et al. Dendritic Cells Coordinate the Development and Homeostasis of Organ-Specific Regulatory T Cells. Immunity. 2016; 44(4):847-59.
38. Delong T, Wiles T A, Baker R L, Bradley B, Barbour G, Reisdorph R, Armstrong M, Powell R L, Reisdorph N, Kumar N, et al. Pathogenic C D4 T cells in type 1 diabetes recognize epitopes formed by peptide fusion. Science. 2016; 351(6274):711-4.
39. Trudeau J D, Kelly-Smith C, Verchere C B, Elliott J F, Dutz J P, Finegood D T, Santamaria P, and Tan R. Prediction of spontaneous autoimmune diabetes in NOD mice by quantification of autoreactive T cells in peripheral blood. The Journal of clinical investigation. 2003; 111(2):217-23.
40. Martinez R J, Zhang N, Thomas S R, Nandiwada S L, Jenkins M K, Binstadt B A, and Mueller D L. Arthritogenic self-reactive CD4+ T cells acquire an FR4hiCD73hi anergic state in the presence of Foxp3+ regulatory T cells. Journal of immunology. 2012; 188(1):170-81.
41. Kalekar L A, Schmiel S E, Nandiwada S L, Lam W Y, Barsness L O, Zhang N, Stritesky G L, Malhotra D, Pauken K E, Linehan J L, et al. CD4(+) T cell anergy prevents autoimmunity and generates regulatory T cell precursors. Nature immunology. 2016; 17(3):304-14.
42. Thornton A M, Korty P E, Tran D Q, Wohlfert E A, Murray P E, Belkaid Y, and Shevach E M. Expression of Helios, an Ikaros transcription factor family member, differentiates thymic-derived from peripherally induced Foxp3+T regulatory cells. Journal of immunology. 2010; 184(7):3433-41.
43. Lu L, Barbi J, and Pan F. The regulation of immune tolerance by FOXP3. Nature reviews Immunology. 2017; 17(11):703-17.
44. Kornete M, Sgouroudis E, and Piccirillo C A. ICOS-dependent homeostasis and function of Foxp3+ regulatory T cells in islets of nonobese diabetic mice. Journal of immunology. 2012; 188(3):1064-74.
45. Wyss L, Stadinski B D, King C G, Schallenberg S, McCarthy N I, Lee J Y, Kretschmer K, Terracciano L M, Anderson G, Surh C D, et al. Affinity for self antigen selects Treg cells with distinct functional properties. Nature immunology. 2016; 17(9):1093-101.
46. Herman A E, Freeman G J, Mathis D, and Benoist C. CD4+CD25+T regulatory cells dependent on ICOS promote regulation of effector cells in the prediabetic lesion. The Journal of experimental medicine. 2004; 199(11):1479-89.
47. Wing K, Onishi Y, Prieto-Martin P, Yamaguchi T, Miyara M, Fehervari Z, Nomura T, and Sakaguchi S. CTLA-4 control over Foxp3+ regulatory T cell function. Science. 2008; 322(5899):271-5.
48. Li H, and Shi B. Tolerogenic dendritic cells and their applications in transplantation. Cellular & molecular immunology. 2015; 12(1):24-30.
49. Tokita D, Mazariegos G V, Zahorchak A F, Chien N, Abe M, Raimondi G, and Thomson A W. High P D-L1/CD86 ratio on plasmacytoid dendritic cells correlates with elevated T-regulatory cells in liver transplant tolerance. Transplantation. 2008; 85(3):369-77.
50. Ben Nasr M, Tezza S, D'Addio F, Mameli C, Usuelli V, Maestroni A, Corradi D, Belletti S, Albarello L, Becchi G, et al. PD-L1 genetic overexpression or pharmacological restoration in hematopoietic stem and progenitor cells reverses autoimmune diabetes. Science translational medicine. 2017; 9(416).
51. Zhang M, Racine J J, Lin Q, Liu Y, Tang S, Qin Q, Qi T, Riggs A D, and Zeng D. MHC-mismatched mixed chimerism restores peripheral tolerance of noncross-reactive autoreactive T cells in NOD mice. Proceedings of the National Academy of Sciences. 2018; 115(10):E2329-E37.
52. Keir M E, Liang S C, Guleria I, Latchman Y E, Qipo A, Albacker L A, Koulmanda M, Freeman G J, Sayegh M H, and Sharpe A H. Tissue expression of PD-L1 mediates peripheral T cell tolerance. The Journal of experimental medicine. 2006; 203(4):883-95.
53. Dendrou C A, Petersen J, Rossjohn J, and Fugger L. HLA variation and disease. Nature reviews Immunology. 2018; 18(5):325-39.
54. Gutierrez-Arcelus M, Rich S S, and Raychaudhuri S. Autoimmune diseases—connecting risk alleles with molecular traits of the immune system. Nature reviews Genetics. 2016; 17(3):160-74.
55. Atkinson M A, Eisenbarth G S, and Michels A W. Type 1 diabetes. The Lancet. 2014; 383(9911):69-82.
56. Wu L, Li N, Zhang M, Xue S L, Cassady K, Lin Q, Riggs A D, and Zeng D. MHC-mismatched mixed chimerism augments thymic regulatory T-cell production and prevents relapse of EAE in mice. Proc Natl Acad Sci USA. 2015; 112(52):15994-9.
57. Balakrishnan A, and Morris G P. The highly alloreactive nature of dual TCR T cells. Current opinion in organ transplantation. 2016; 21(1):22-8.
58. Millar D G, and Ohashi P S. Central tolerance: what you see is what you don't get!Nature immunology. 2016; 17(2):115-6.
59. Schneider A, Rieck M, Sanda S, Pihoker C, Greenbaum C, and Buckner J H. The effector T cells of diabetic subjects are resistant to regulation via CD4+ FOXP3+ regulatory T cells. Journal of immunology. 2008; 181(10):7350-5.
60. Ferreira C, Singh Y, Furmanski A L, Wong F S, Garden O A, and Dyson J. Non-obese diabetic mice select a low-diversity repertoire of natural regulatory T cells. Proceedings of the National Academy of Sciences of the United States of America. 2009; 106(20):8320-5.
61. Okubo Y, Torrey H, Butterworth J, Zheng H, and Faustman D L. Treg activation defect in type 1 diabetes: correction with TNFR2 agonism. Clinical & translational immunology. 2016; 5(1):e56.
62. D'Alise A M, Auyeung V, Feuerer M, Nishio J, Fontenot J, Benoist C, and Mathis D. The defect in T-cell regulation in NOD mice is an effect on the T-cell effectors. Proceedings of the National Academy of Sciences of the United States of America. 2008; 105(50):19857-62.
63. Amarnath S, Mangus C W, Wang J C, Wei F, He A, Kapoor V, Foley J E, Massey P R, Felizardo T C, Riley J L, et al. The PDL1-PD1 axis converts human TH1 cells into regulatory T cells. Science translational medicine. 2011; 3(111):111 ra20.
64. Stathopoulou C, Gangaplara A, Mallett G, Flomerfelt F A, Liniany L P, Knight D, Samsel L A, Berlinguer-Palmini R, Yim J J, Felizardo T C, et al. PD-1 Inhibitory Receptor Downregulates Asparaginyl Endopeptidase and Maintains Foxp3 Transcription Factor Stability in Induced Regulatory T Cells. Immunity. 2018; 49(2):247-63 e7.
65. Ellestad K K, Thangavelu G, Ewen C L, Boon L, and Anderson C C. PD-1 is not required for natural or peripherally induced regulatory T cells: Severe autoimmunity despite normal production of regulatory T cells. European journal of immunology. 2014; 44(12):3560-72.
66. Yi T, Li X, Yao S, Wang L, Chen Y, Zhao D, Johnston H F, Young J S, Liu H, Todorov I, et al. Host APCs augment in vivo expansion of donor natural regulatory T cells via B7H1/B7.1 in allogeneic recipients. Journal of immunology. 2011; 186(5):2739-49.
67. Zuber J, and Sykes M. Mechanisms of Mixed Chimerism-Based Transplant Tolerance. Trends Immunol. 2017; 38(11):829-43.
68. Spinner M A, Fernandez-Vina M, Creary L E, Quinn O, Elder L, Arai S, Johnston L J, Meyer E H, Miklos D B, Muffly L S, et al. HLA-mismatched unrelated donor transplantation using TLI-ATG conditioning has a low risk of GVHD and potent antitumor activity. Blood advances. 2017; 1(17):1347-57.

1. A method of treating or preventing the onset of an autoimmune disease in a subject, comprising administering to the subject radiation-free, non-myeloablative low doses of cyclophosphamide (CY), pentostatin (PT), and anti-thymocyte globulin (ATG), and administering to the subject a population of CD4⁺ T-depleted hematopoietic cells from a donor.

2. A method of inducing haploidentical mixed chimerism in a subject, comprising administering to the subject radiation-free, non-myeloablative low doses of CY, PT and ATG, and administering to the subject a population of CD4⁺ T-depleted hematopoietic cells from a donor.

3. The method of claim 2, wherein the donor CD4⁺ T-depleted hematopoietic cells include donor CD4⁺ T-depleted spleen cells, and donor CD4⁺ T-depleted bone marrow cells.

4. The method of claim 2, wherein the donor CD4⁺ T-depleted hematopoietic cells are CD4⁺ T-depleted G-CSF-mobilized blood mononuclear cells comprising donor hematopoietic stem cells and CD8⁺ T cells.

5. The method of claim 4, wherein the donor is haploidentical to the subject.

6. The method of claim 4, wherein the donor is haplo-mismatched to the subject.

7. The method of claim 4, wherein the donor is not full-HLA- or MHC-matched to the subject.

8. The method of claim 7, wherein the subject is a mammal.

9. The method of claim 8, wherein the subject is human.

10. The method of claim 9, wherein the subject suffers from or at an elevated risk of suffering from an autoimmune disease selected from the group consisting of type 1 diabetes, multiple sclerosis, systemic lupus, scleroderma, and chronic graft versus host disease, aplastic anemia, and arthritis.

11. A conditioning regimen for inducing haploidentical mixed chimerism in a subject comprising administration of radiation-free, non-myeloablative low doses of CY, PT, and ATG, and administration of a population of CD4⁺ T-depleted hematopoietic cells from a donor.

12. The conditioning regimen of claim 11, wherein the donor CD4⁺ T-depleted hematopoietic cells include donor CD4⁺ T-depleted spleen cells, and donor CD4⁺ T-depleted bone marrow cells.

13. The conditioning regimen of claim 12, wherein the donor CD4⁺ T-depleted hematopoietic cells are CD4⁺ T-depleted G-CSF-mobilized blood mononuclear cells comprising donor hematopoietic stem cells and CD8⁺ T cells.

14. The conditioning regimen of claim 13, wherein the donor is haploidentical to the subject.

15. The conditioning regimen of claim 13, wherein the donor is haplo-mismatched to the subject.

16. The conditioning regimen of claim 13, wherein the donor is not full-HLA- or MHC-matched to the subject.

17. The conditioning regimen of claim 16, wherein the subject is a mammal.

18. The conditioning regimen of claim 17, wherein the subject is human.

19. The method of claim 1, wherein the donor CD4⁺ T-depleted hematopoietic cells include donor CD4⁺ T-depleted spleen cells, and donor CD4⁺ T-depleted bone marrow cells.

20. The method of claim 1, wherein the donor is haploidentical, haplo-mismatched, or is not full-HLA- or MHC-matched to the subject.
